SEQUENCE LISTING (5 sequences)

SEQ ID NO: 1 - DNA, 3669 bp, Artificial Sequence
  Artificial Oligonucleotide Encoding Modified Spike Protein of SARS-CoV-2
atgtttgtctttctggtcctgctgcctctggtctcctcacagtgcgtgaatctgactacccgaactcagctgccccctgcttacaccaacagcttcaccaggggcgtgtactatcctgacaaggtgtttcggtccagcgtgctgcactctacacaggatctgtttctgccattcttttccaacgtgacctggttccacgctatccacgtgagcggcaccaatggcacaaagaggttcgacaatcccgtgctgccttttaacgatggcgtgtatttcgcctctaccgagaagtccaacatcatccggggctggatctttggcaccacactggacagcaagacacagtctctgctgatcgtgaacaatgctaccaacgtggtcatcaaggtgtgcgagttccagttttgtaatgatcccttcctgggcgtgtactatcataagaacaataagtcctggatggagagcgagtttcgggtgtattcttccgccaacaactgcacatttgagtacgtgagccagcctttcctgatggacctggagggcaagcagggcaatttcaagaacctgagagagttcgtgtttaagaatatcgacggctacttcaaaatctactctaagcacacccccatcaacctggtgcgcgacctgcctcagggcttctccgccctggagcccctggtggatctgcctatcggcatcaacatcaccaggtttcagacactgctggccctgcatcggtcttacctgacacccggcgacagctcttccggatggaccgctggcgccgctgcctactatgtgggctacctccagcctagaaccttcctgctgaagtacaacgagaatggcaccatcacagacgctgtggattgcgccctggaccccctgtccgagacaaagtgtacactgaagagctttaccgtggagaagggcatctatcagacaagcaatttcagagtgcagccaaccgagtctatcgtgcgctttcctaatatcacaaacctgtgcccatttggcgaggtgttcaacgctacccgcttcgcctccgtgtacgcctggaatagaaagaggatctccaactgcgtggccgactattccgtgctgtacaactccgcctccttctccaccttcaagtgctatggcgtgagcccaacaaagctgaatgacctgtgctttaccaacgtctacgctgattctttcgtgatcagaggcgacgaggtgcgccagatcgcccccggccagacaggcaagatcgctgactacaattataagctgccagacgatttcaccggctgcgtgatcgcctggaactccaacaatctggatagcaaagtgggcggcaactacaattatctgtacaggctgtttcggaagtccaatctgaagcccttcgagagagacatctctacagaaatctaccaggctggctccaccccttgcaatggcgtggagggctttaactgttatttcccactccagagctacggcttccagcccacaaacggcgtgggctatcagccttaccgcgtggtggtgctgtcctttgagctgctgcacgctccagctacagtgtgcggccccaagaagagcaccaatctggtgaagaacaagtgcgtgaacttcaacttcaacggcctgaccggcacaggcgtgctgaccgagtccaacaagaagttcctgccatttcagcagttcggcagagacatcgccgataccacagacgctgtgcgcgacccacagaccctggagatcctggacatcacaccctgctctttcggcggcgtgtccgtgatcacacccggcaccaatacaagcaaccaggtggccgtgctgtatcaggacgtgaattgtaccgaggtgcctgtggctatccacgccgatcagctgaccccaacatggagggtgtactccaccggcagcaacgtcttccagacacgggccggatgtctgatcggagctgagcatgtgaacaatagctatgagtgcgacatcccaatcggcgccggcatctgtgcttcttaccagacccagacaaactctcccaggagggccaggtccgtggcctcccagtctatcatcgcctataccatgagcctgggcgccgagaactccgtggcttactctaacaattccatcgctatcccaaccaacttcacaatctctgtgaccacagagatcctgcccgtgagcatgaccaagacatctgtggactgcacaatgtatatctgtggcgattctaccgagtgctccaacctgctgctccagtacggctccttttgtacccagctgaatagagccctgacaggcatcgctgtggagcaggataagaacacacaggaggtgttcgcccaggtgaagcaaatctacaagaccccccctatcaaggactttggcggcttcaatttttcccagatcctgcctgatccaagcaagccctctaagaggtcctttatcgaggacctgctgttcaacaaggtgaccctggctgatgccggcttcatcaagcagtatggcgattgcctgggcgacatcgctgccagggacctgatctgcgcccagaagtttaatggcctgaccgtgctgccacccctgctgacagatgagatgatcgctcagtacacatccgctctgctggccggcaccatcacaagcggatggaccttcggcgctggagccgccctccagatcccttttgccatgcagatggcttatagattcaacggcatcggcgtgacccagaatgtgctgtacgagaaccagaagctgatcgccaatcagtttaactctgctatcggcaagatccaggactccctgtcctccaccgcctccgccctgggcaagctccaggatgtggtgaatcagaacgctcaggccctgaataccctggtgaagcagctgtccagcaacttcggcgccatctcttccgtgctgaatgacatcctgtccaggctggacaaggtggaggctgaggtgcagatcgacaggctgatcaccggcaggctccagtccctccagacctatgtgacacagcagctgatcagagctgccgagatccgcgcttccgccaatctggctgccaccaagatgagcgagtgcgtgctgggccagtctaagcgggtggacttttgtggcaagggctatcacctgatgagcttccctcagtctgccccacacggcgtggtgtttctgcatgtgacctacgtgcccgctcaggagaagaacttcaccacagctcctgccatctgccacgatggcaaggcccattttccaagagagggcgtgttcgtgagcaacggcacccattggtttgtgacacagcgcaatttctacgagccccagatcatcaccacagacaacaccttcgtgtccggcaactgtgacgtggtcatcggcatcgtgaacaataccgtgtatgatccactccagcccgagctggactcctttaaggaggagctggataagtatttcaagaatcataccagccctgacgtggatctgggcgacatctccggcatcaatgccagcgtggtgaacatccagaaggagatcgaccgcctgaacgaggtggctaagaatctgaacgagagcctgatcgacctccaggagctgggcaagtatgagcagtacatcaagtggccccgcagaagacggcggagacggagaagataa

SEQ ID NO: 2 - protein, 1222 aa, Artificial Sequence
  Modified Oligonucleotide of SARS-CoV-2 Spike Protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPRRRRRRRRR

SEQ ID NO: 3 - DNA, 24 bp, Artificial Sequence
  Forward Primer
acaggtacgttaatagttaatagc

SEQ ID NO: 4 - DNA, 20 bp, Artificial Sequence
  Reverse PCR Primer
atattgcagcagtacgcaca

SEQ ID NO: 5 - DNA, 28 bp, Artificial Sequence
  FAM-labeled PCR Probe
  misc_signal (1)..(1): 1-position is covalently linked to FAM-fluorophore
  misc_signal (28)..(28): 28-position is covalently attached to TAMRA quencher
acactagccatccttactgcgcttcgbb

(19) United States
(12) Patent Application Publication
(10) Pub. No.: US 2022/0356212 A1
(43) Pub. Date: Nov. 10, 2022

(54) MODIFIED SPIKE PROTEIN AND METHOD OF TREATMENT

(71) Applicant: Nanogen Pharmaceutical Biotechnology JSC, Ho Chi Minh (VN)

(72) Inventors: Nhan Ho, Ho Chi Minh City (VN); Si Minh Do, Ho Chi Minh City (VN); Bruce May, Ho Chi Minh City (VN)

(73) Assignee: Nanogen Pharmaceutical Biotechnology JSC, Ho Chi Minh City (VN)

(21) Appl. No.: 17/705,090

(22) Filed: Mar. 25, 2022

Related U.S. Application Data
(60) Provisional application No. 63/257,751, filed on Oct. 20, 2021.

(30) Foreign Application Priority Data
Mar. 26, 2021 (VN) 1-2021-01629

(51) Int. Cl. C07K 14/005; A61K 39/215; A61P 31/14

(52) U.S. Cl. CPC C07K 14/005; A61K 39/215; A61P 31/14; C12N 2770/20022; C12N 2770/20034; C12N 2770/20071; A61K 2039/545

(57) ABSTRACT

In one embodiment, the present specification discloses a modified spike (S) protein comprising of SEQ ID NO: 2, the modified spike protein encoded by a gene sequence of SEQ ID NO: 1, and methods of using the modified spike protein as disclosed herein.

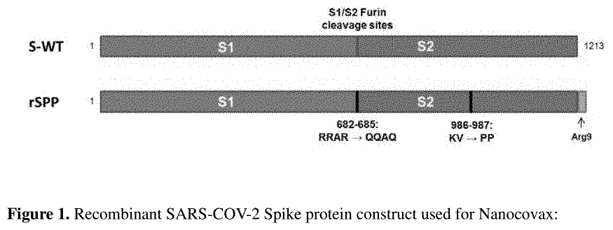

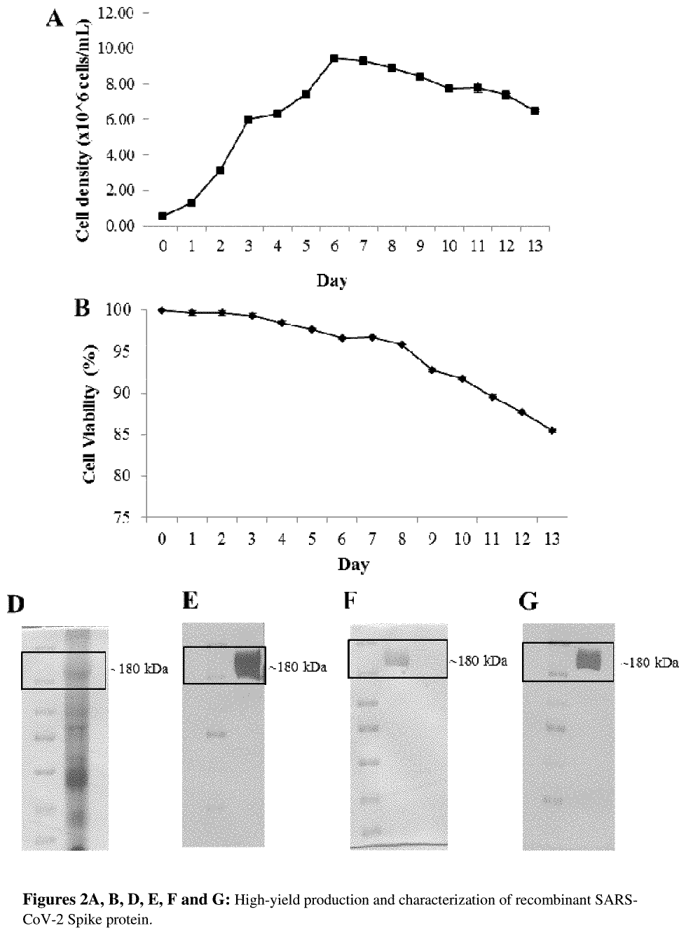

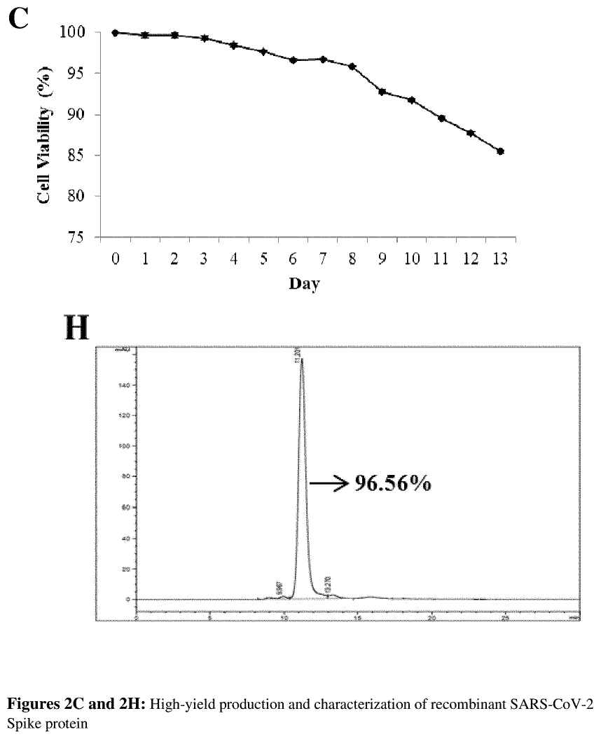

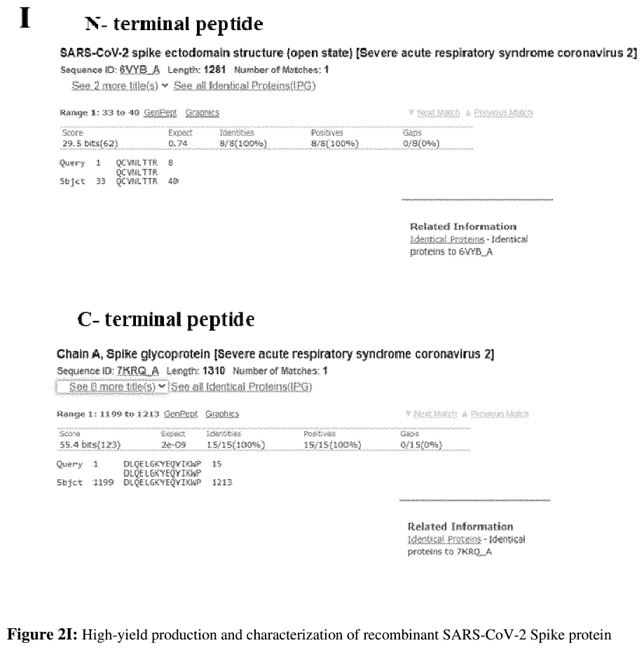

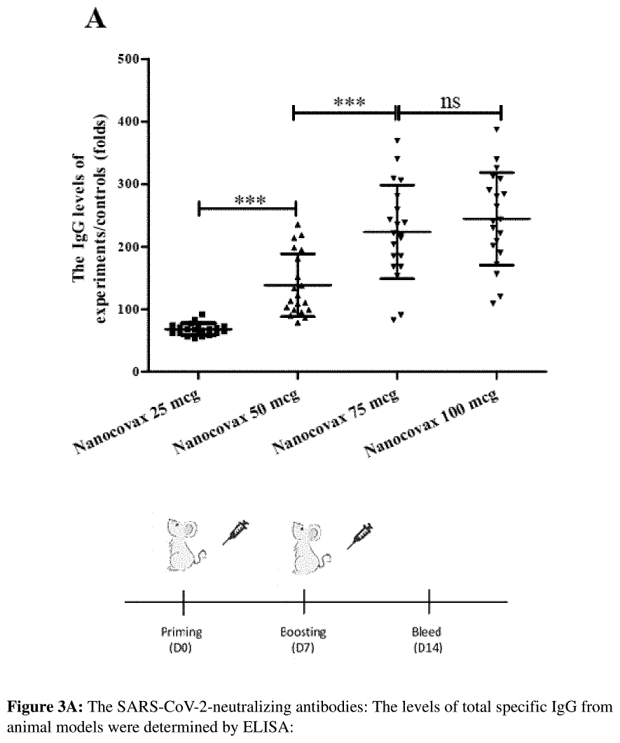

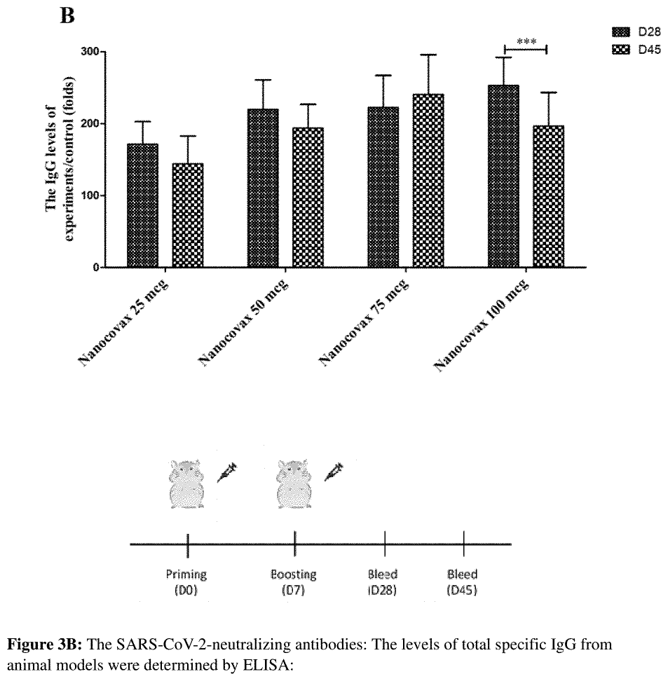

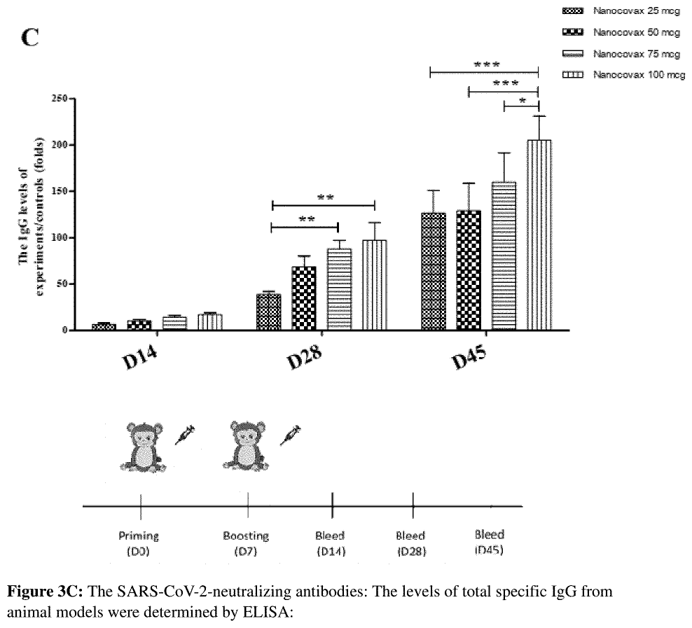

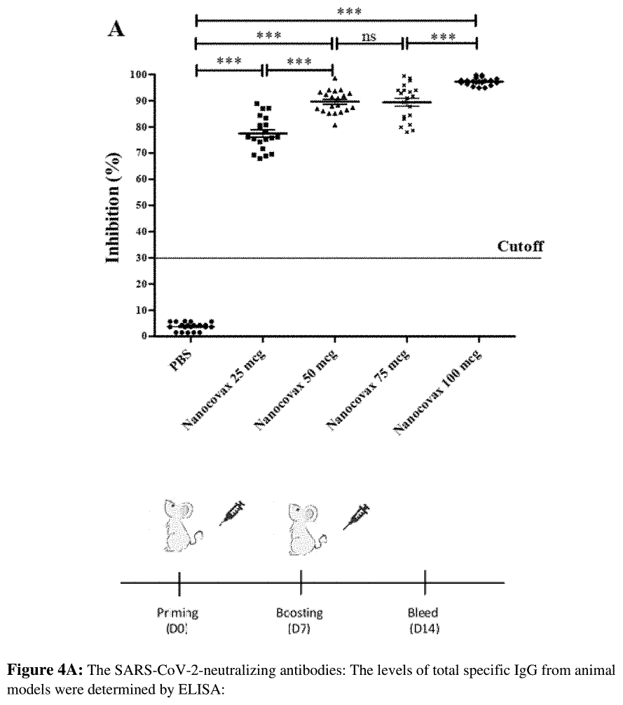

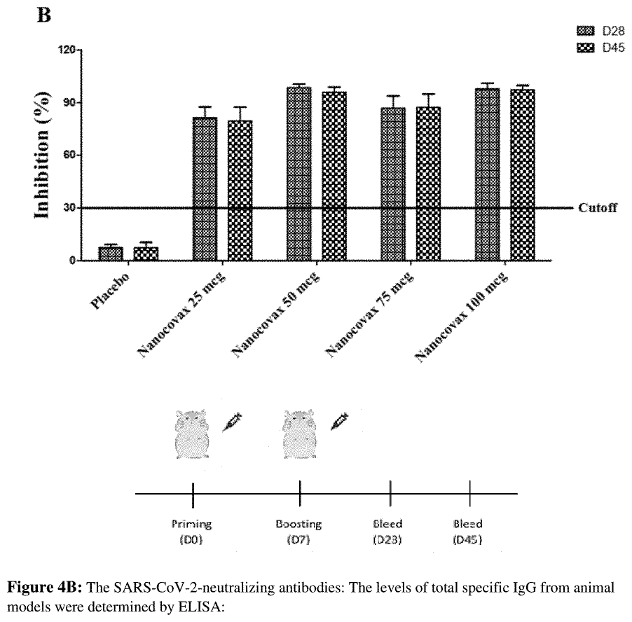

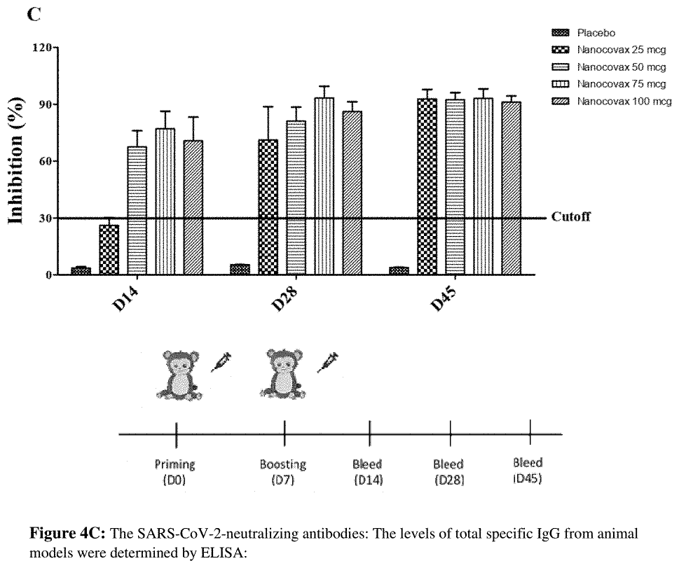

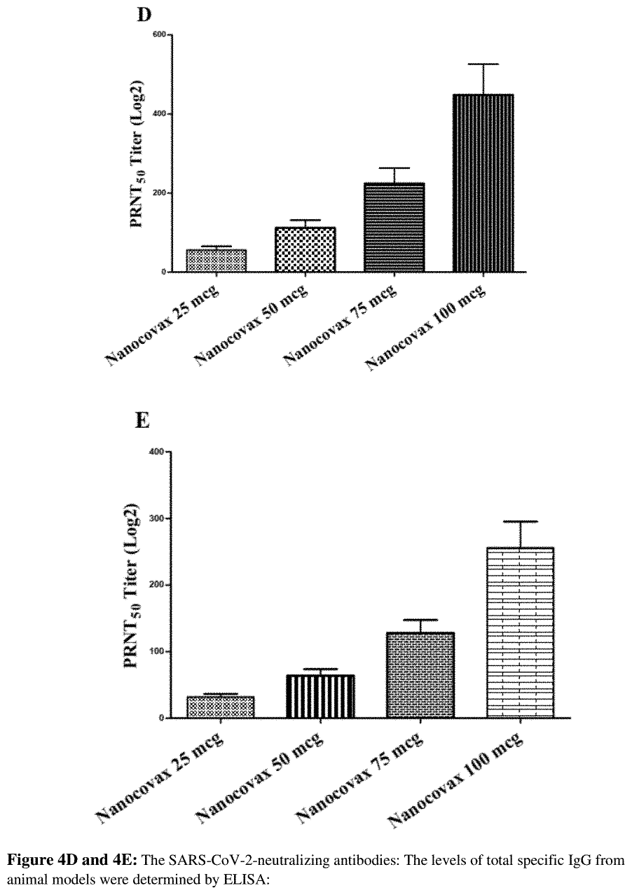

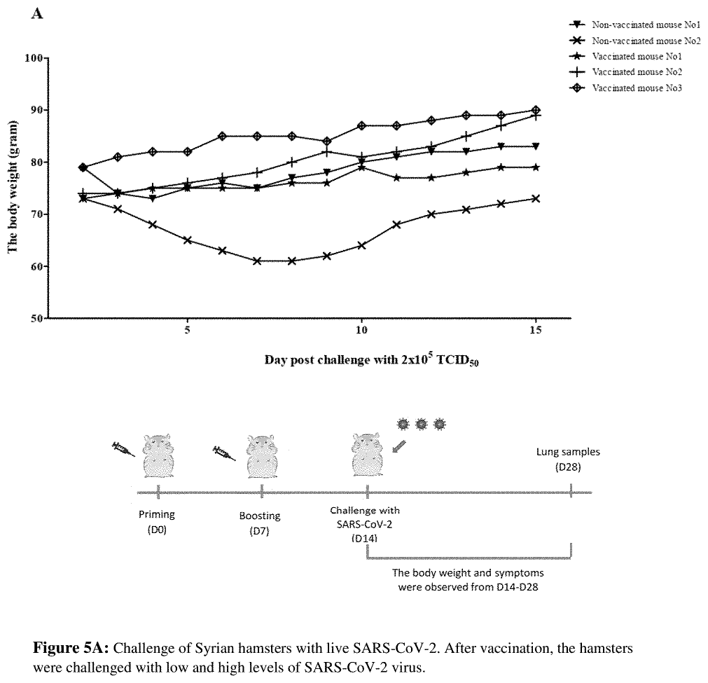

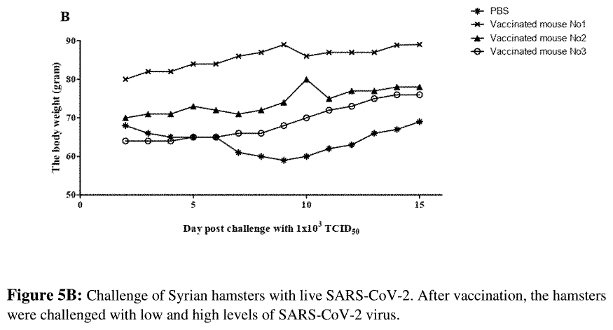

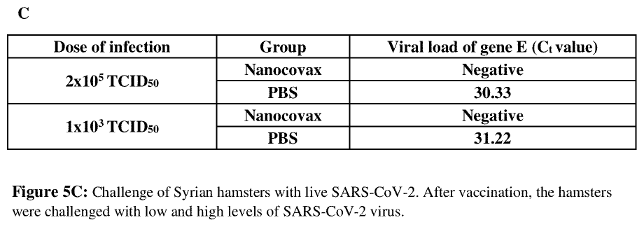

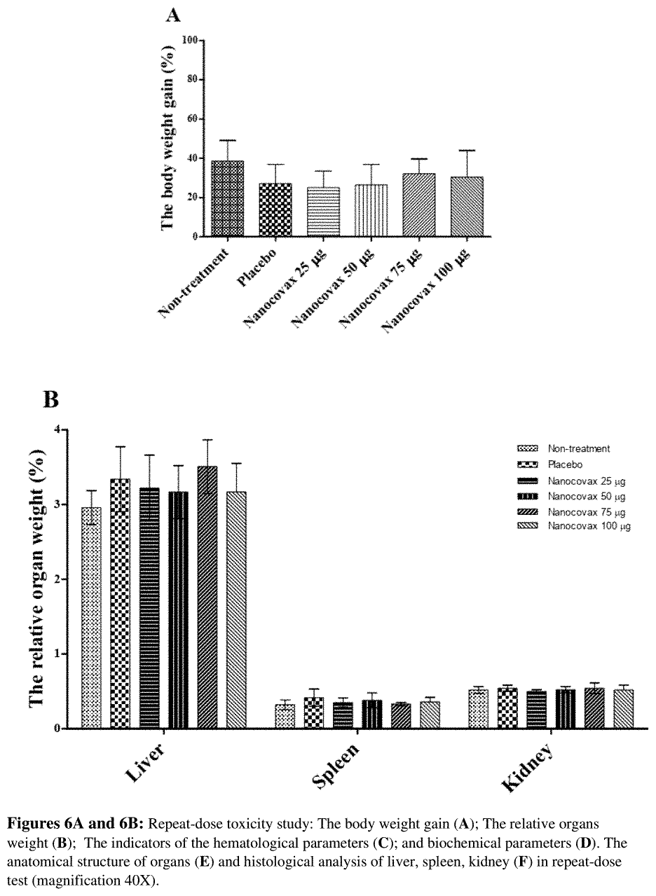

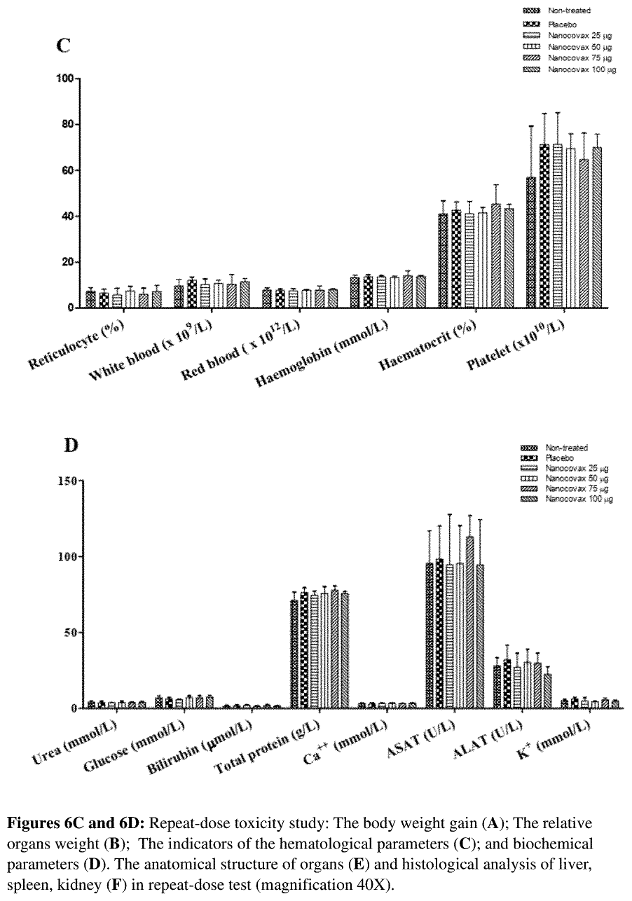

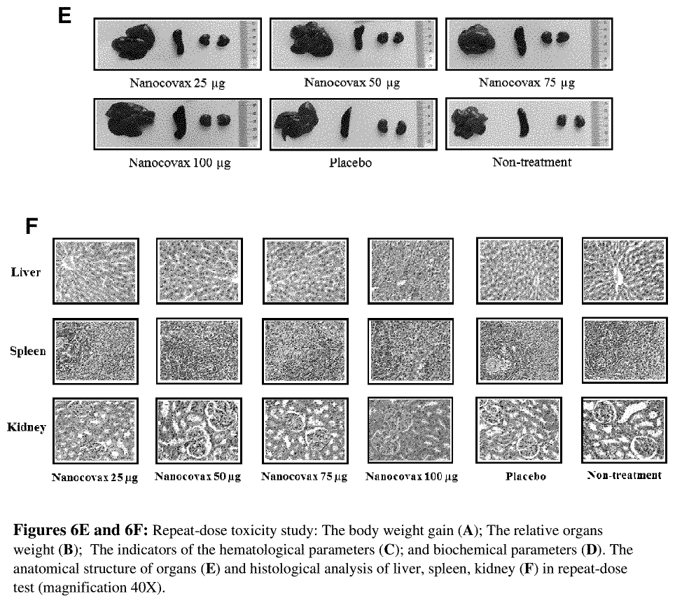

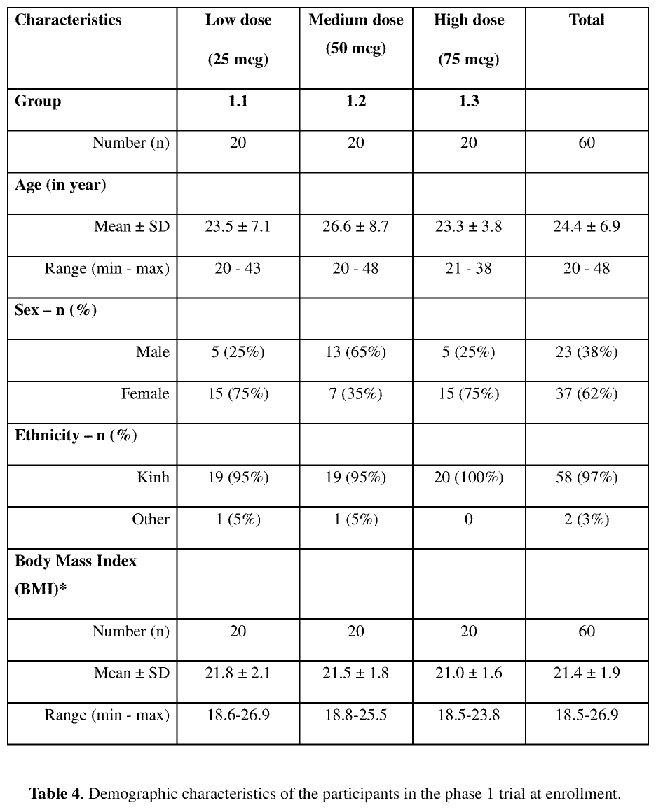

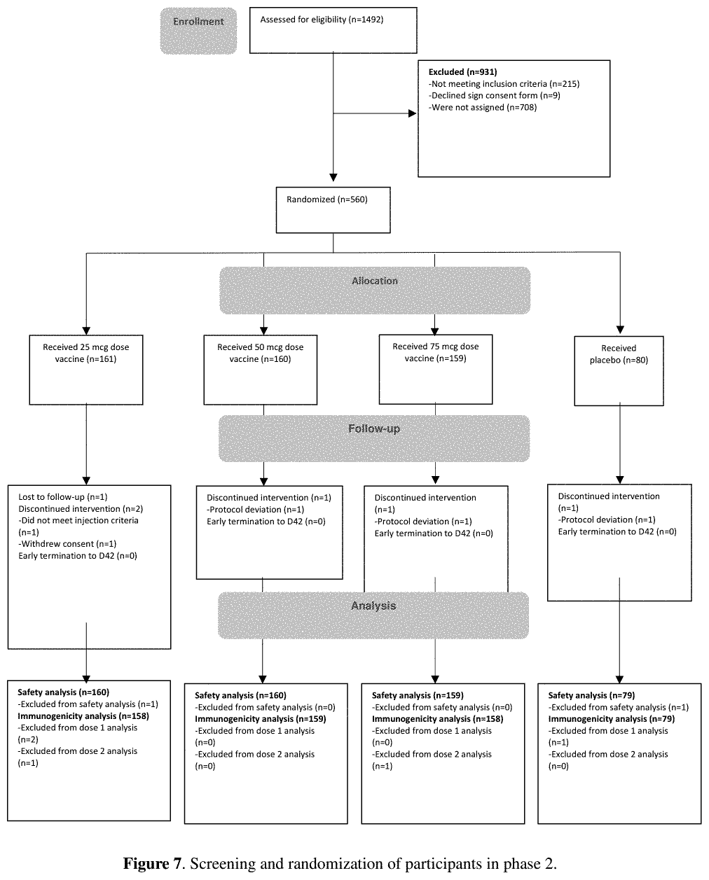

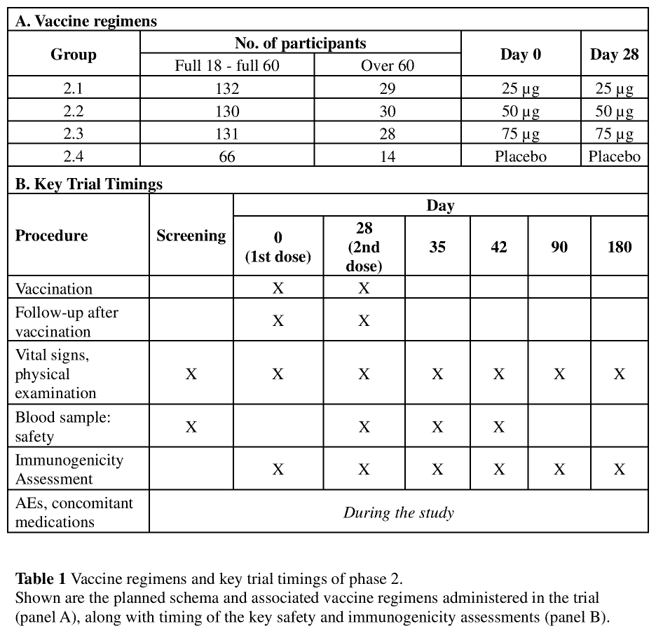

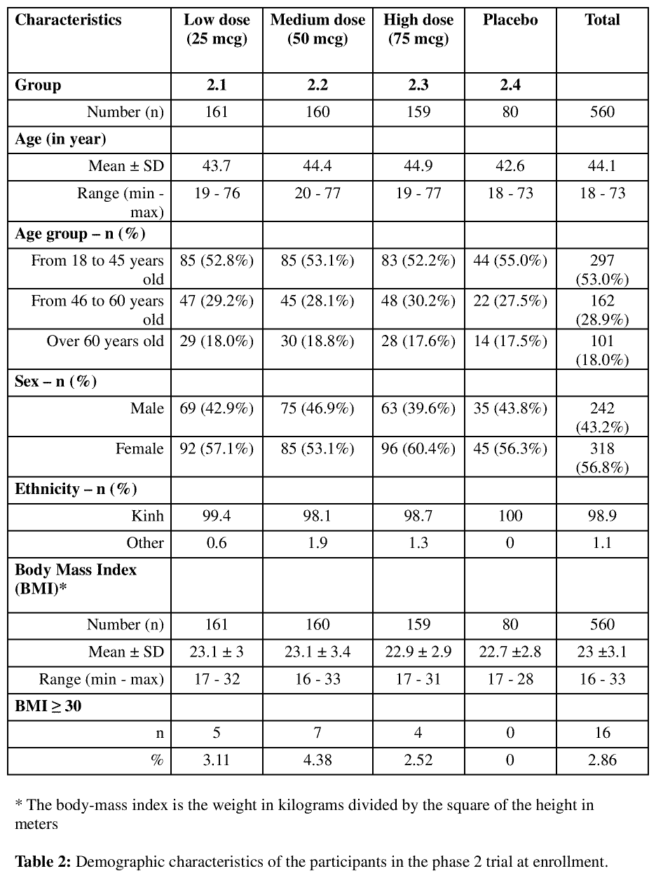

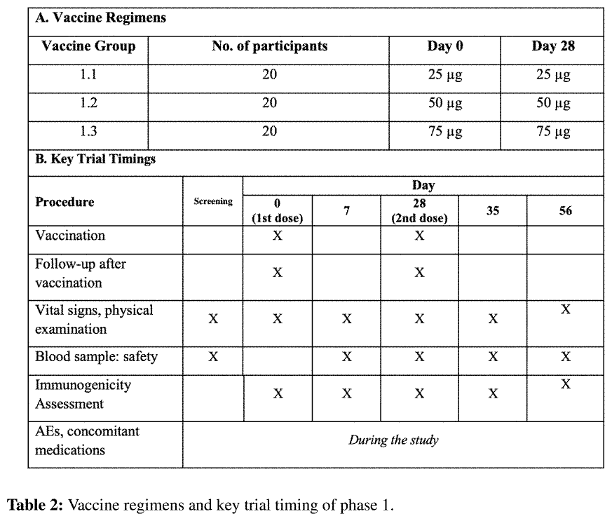

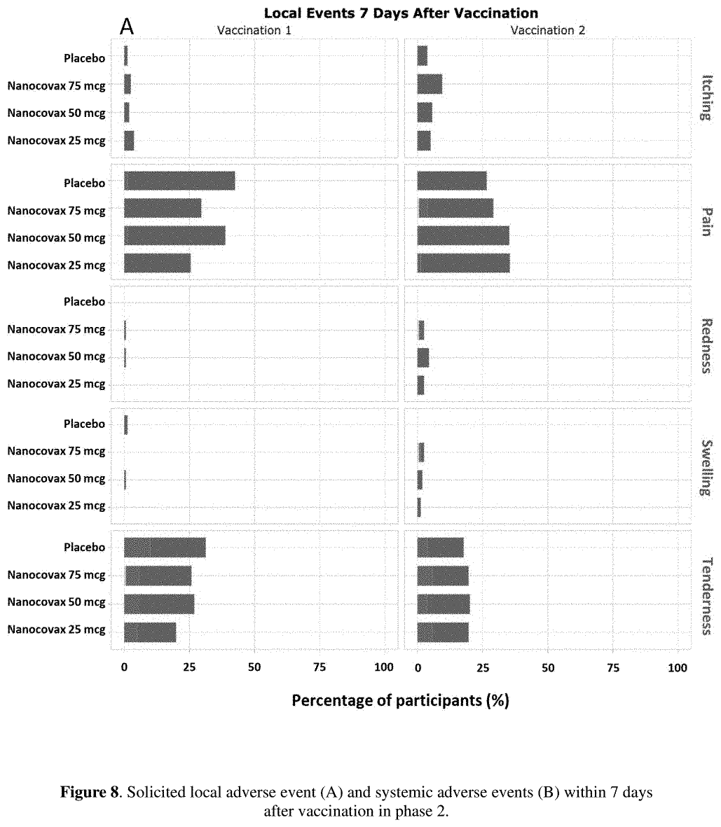

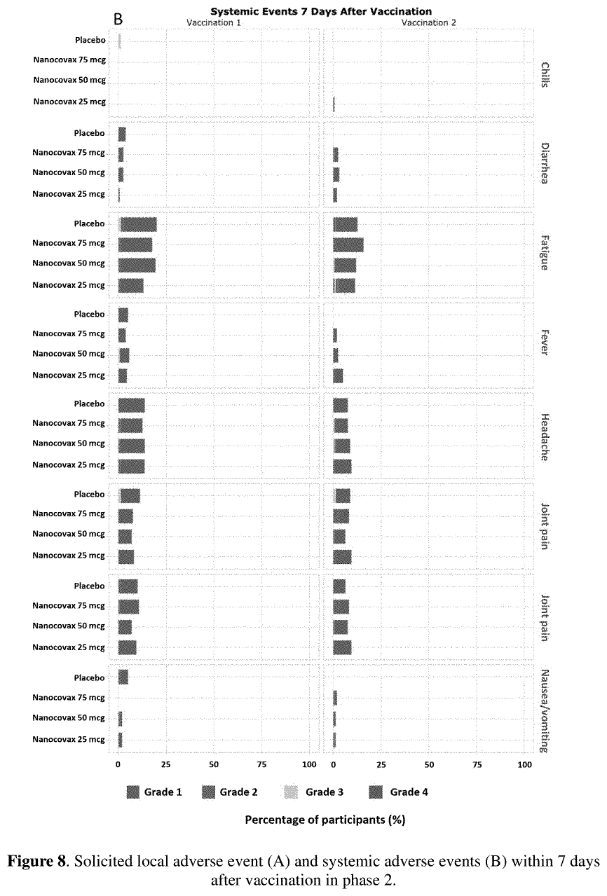

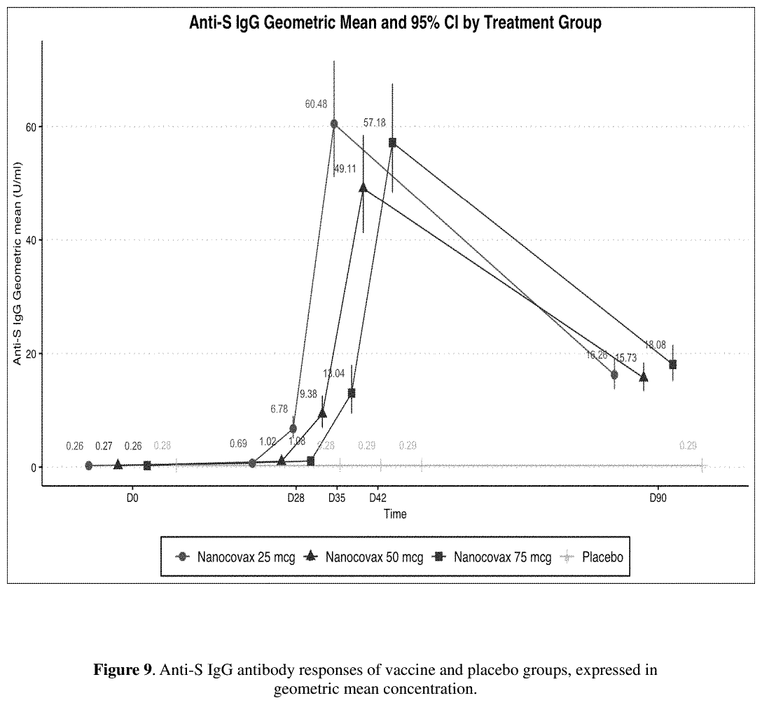

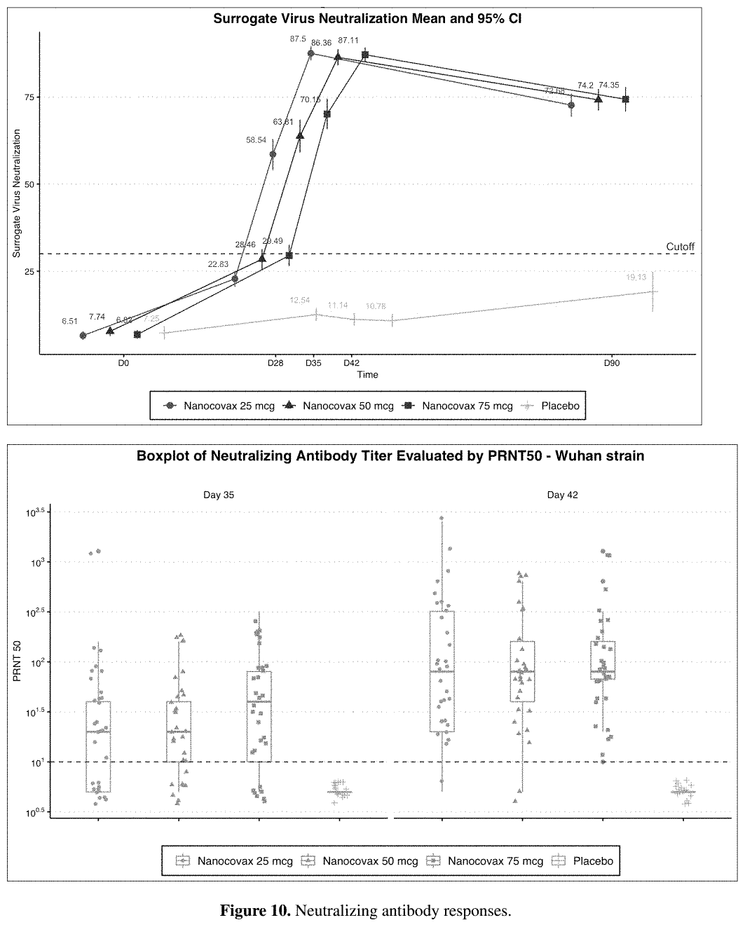

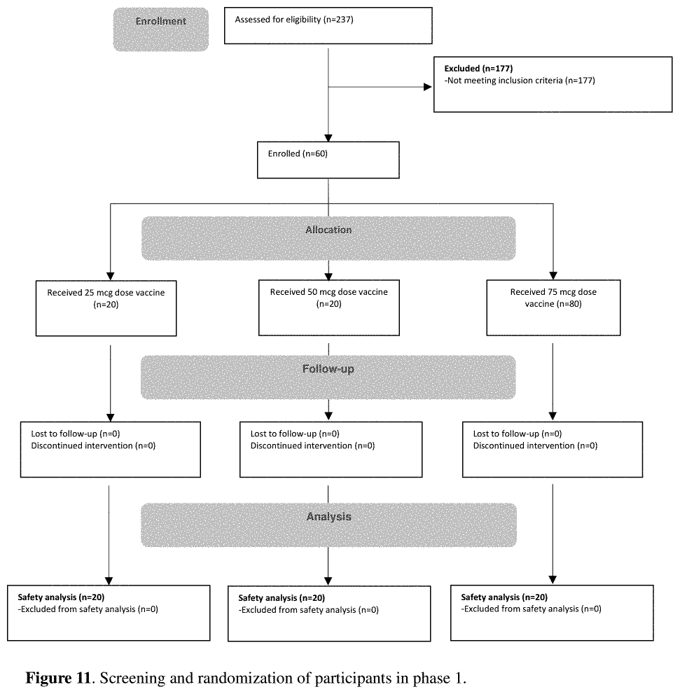

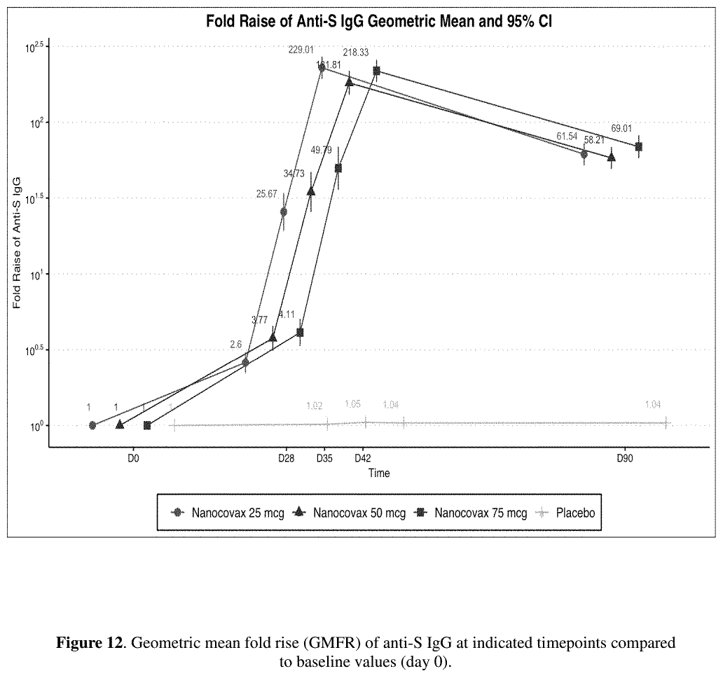

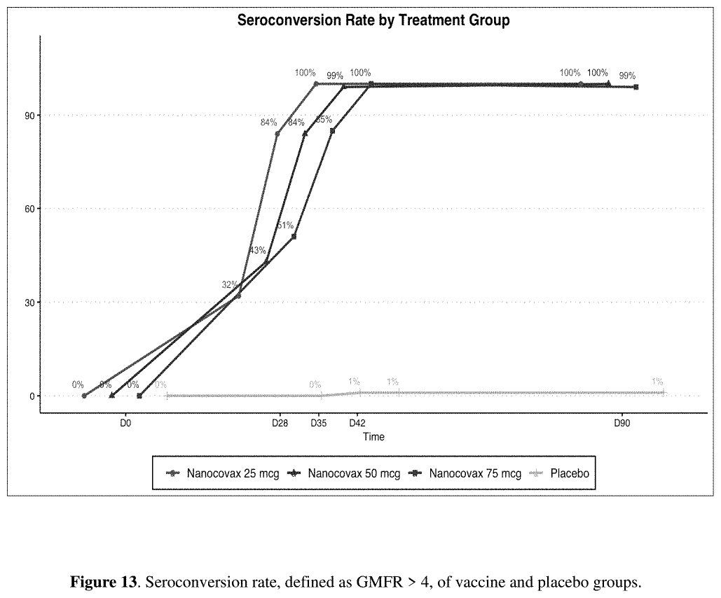

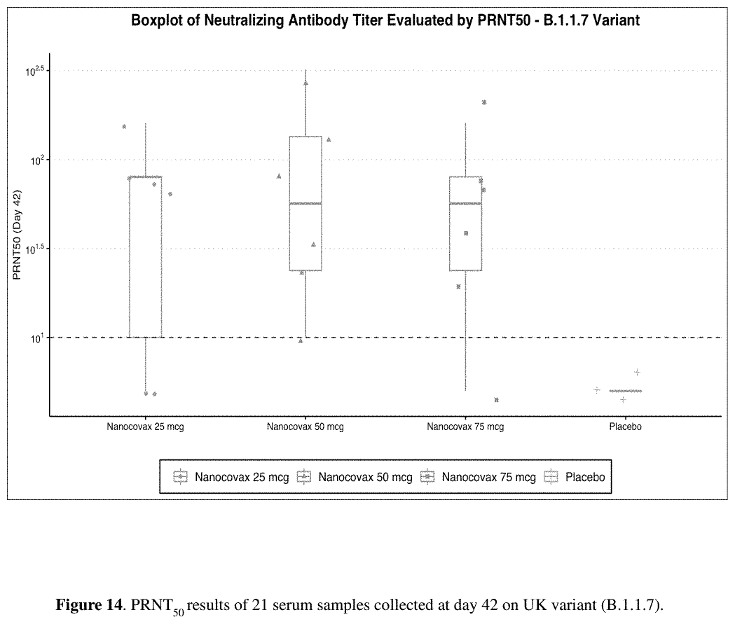

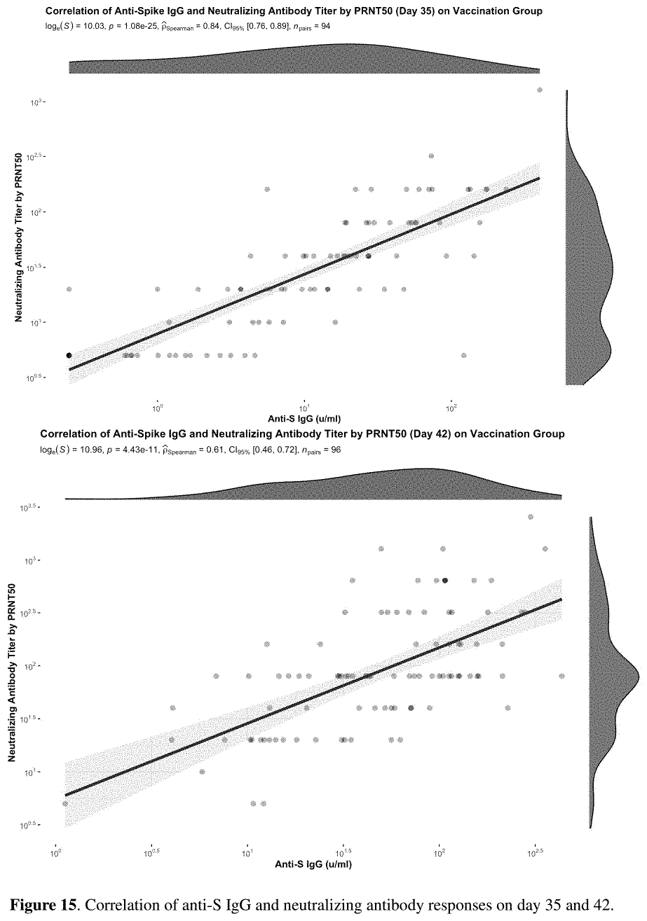

### CROSS-REFERENCE TO RELATED APPLICATIONS

This application claims priority to Vietnamese Patent Application No. 1-2021-01629 filed on Mar. 26, 2021, and claims the benefit under 35 USC 119(e) of Application No. 63/257,751 filed Oct. 20, 2021, the entire contents of which are incorporated into this application by reference.

### BACKGROUND OF THE INVENTION

The Coronavirus disease-2019 (COVID-19) pandemic caused by the Severe Acute Respiratory Syndrome coronavirus 2 (SARS-CoV-2), has become a dire global health emergency. Since it was first reported in Wuhan, China at the end of 2019, as of July 2021, more than 177 million cases and over 4 million deaths due to Covid-19 have been reported worldwide. WHO. WHO Coronavirus Dashboard. Https://Covid19WhoInt/2021:1.

SARS-CoV-2 is a member betacoronavirus, named for its corona of spike (S) proteins protruding from the viral envelope. Hu B. et al. Characteristics of SARS-CoV-2 and COVID-19. Nature Reviews Microbiology 2021; 19:141-54. http://doi.org/10.1038/s41579_ 020-00459-7. SARS-CoV-2 S, a heavily glycosylated protein, is responsible for the attachment to angiotensin-converting enzyme (ACE2) which helps the virus entry to host cells in human and animals. SARS-CoV-2 S glycoprotein is the antigen of choice for Covid-19 vaccine development due to its highly antigenic property.

Like SARS-CoV (79% genomic sequence identity), SARS-CoV-2 utilizes the receptor angiotensin-converting enzyme 2 (ACE-2), which is expressed on numerous cells including lung, heart, kidney and intestine cells as the entry fusion receptor by its viral spike, a homotrimeric complex of spike (S) proteins. The S protein is a homomeric class I fusion protein with each S monomer containing the N-terminal S1 subunit, which includes the receptor-binding domain (RBD), and the C-terminal S2 subunit, which is anchored to the viral membrane and is required for trimerization of the spike itself and fusion to host membrane. During fusion to host cell membranes, the S protein undergoes extensive conformational changes that cause dissociation of the S1 subunit from the complex and the formation of a stable post-fusion conformation of the S2 subunit. Therefore, the S protein of SARS-CoV-2 plays a vital role in the invasive process.

Potential vaccines against SARS-CoV-2 have therefore focused on the S protein and include mRNA-lipid nanoparticles that encode the S protein, viral vectored DNA-based vaccines (notably recombinant adenoviruses), and subunit vaccines that contain purified S protein. One report from the World Health Organization (WHO) estimated that 102 S protein-targeted vaccines are in clinical development and 185 S protein-targeted vaccines are in pre-clinical development (COVID-19 Vaccine Tracker and Landscape). The vaccine candidates that have been developed or are under development include recombinant protein vaccines, whole inactivated vaccines, viral vector-based vaccines, and DNA vaccines to prevent virus infection. Currently, three vaccines are approved by Food & Drug Administration (FDA): BNT162b2 is a lipid nanoparticle-formulated, nucleoside-modified mRNA vaccine (Comirnaty®; Pfizer/BionNtech), Moderna COVID-19 vaccine is mRNA-based, and Janssen (Johnson and Johnson) is based on an adenovirus vector and is used in adults aged 18 years and older. Besides vaccines, oligonucleotides, peptides, interferon and small-molecule drugs have been suggested to control infection of SARS-CoV-2. During a public health emergency, the FDA has approved three anti-SARS-CoV-2 mAb therapies for the treatment of non-hospitalized patients with mild-to-moderate COVID-19 including bamlanivimab, etesevimab or casirivimab with imdevimab.

### SUMMARY OF THE INVENTION

Therefore a continuing need exists for novel, safe and effective vaccines for the treatment of viruses such as SARS-CoV-2. The following embodiments, aspects and variations thereof are exemplary and illustrative are not intended to be limiting in scope.

Nanocovax is a subunit vaccine, developed and manufactured at Nanogen Pharmaceutical Biotechnology JSC. Nanocovax contains a full-length prefusion stabilized recombinant SARS-CoV-2 S glycoproteins and aluminum hydroxide adjuvant. In rodent and monkey models, Nanocovax induced high levels of anti-S antibody (Ab). Neutralizing antibody titers were evaluated by microneutralization on Wuhan strain and surrogate virus neutralization test. Nanocovax conferred a remarkable protection against SARS-CoV-2 infection in hamster challenge model.

In one embodiment, the present application discloses a modified spike (S) protein comprising of SEQ ID NO: 2. In one variation, the modified spike protein or glycoprotein is consisting of SEQ ID NO: 2. In another variation, the modified spike protein or modified polypeptide is an immunogenic protein. In another aspect, the modified spike protein is encoded by a gene sequence of SEQ ID NO: 1.

In another embodiment, there is provided a gene sequence comprising of SEQ ID NO: 1. In one variation, the gene sequence is a polynucleotide comprising of or consisting of SEQ ID NO: 1. In another aspect, the modified spike protein is cloned with a pCNeoMEM vector and expressed by a host cell line CHO-DG44.

In another embodiment, there is provided an immunogenic composition comprising a modified spike (S) protein comprising of SEQ ID NO: 2; and an adjuvant. In one variation, the immunogenic composition comprises a non-naturally occurring sudden acute respiratory syndrome coronavirus 2 (SARS-CoV-2) spike (CoV S) glycoprotein of SEQ ID NO: 2 and an adjuvant. In another aspect of the immunogenic composition, the modified spike protein is cloned with a pCNeoMEM vector and expressed by a host cell line CHO-DG44. In another aspect of the immunogenic composition, the dose comprising the modified spike protein ranges from 5 μg/mL to 100 μg/mL. In one variation, the concentration of the modified spike protein in the composition ranges from about 5 μg/mL to 100 μg/mL, 5 μg/mL to 50 μg/mL, 5 μg/mL to 25 μg/mL, 5 μg/mL to 20 μg/mL or about 5 μg/mL to 10 μg/mL. In another variation, the concentration of the modified spike protein ranges from 1 μg/mL to 300 μg/mL. In another variation, the composition comprises about 3 μg, 5 μg, 7 μg, 9 μg, 10 μg, 15 μg or about 20 μg of the modified spike protein. In another variation, the composition comprises from about 3 μg to about 50 μg of the modified spike protein. In another variation, the composition comprises a salt, such as NaCl, to form an osmotic concentration of 286 mOsmol/kg (milliosmoles per kilogram of water). In another variation, the composition comprises a salt to form an osmotic concentration in the range of 285-295 mOsm/kg H₂O or 285-295 mmol/kg (milliosmole (mOsm)).

In another aspect, the immunogenic composition further comprising a pharmaceutically acceptable buffer. In one variation, the buffer is a phosphate buffer. In one variation, the composition has a pH from 6.0 to 7.0. In another variation, the composition has a pH of 6.3, 6.5, 6.7 or 6.9. In another aspect, the immunogenic composition further comprises an adjuvant. In another aspect, the adjuvant is selected from the group consisting of aluminum phosphate, aluminum hydroxide and saponin QS-21, or a mixture thereof.

In one variation, at least one of the aluminum phosphate and aluminum hydroxide is present at a concentration of 0.2 mg/mL to 1 mg/mL. In another variation, the saponin QS-21 is obtained from the bark of the Quillaja saponaria tree, called QS-21, and is present at a concentration in the range of about 5 μg/mL to 50 μg/mL, 5 μg/mL to 25 μg/mL, 5 μg/mL to 20 μg/mL or about 5 μg/mL to 10 μg/mL.

In another embodiment, the application discloses a method of stimulating an immune response against SARS-CoV-2 or a SARS-CoV-2 variant thereof, in a subject comprising administering a modified spike protein of SEQ ID NO: 2, or a composition comprising a modified spike protein of SEQ ID NO: 2. In one variation, the method comprises the administration of the immunogenic modified spike protein of SEQ ID NO: 2. In one variation, the immunogenic composition comprises a non-naturally occurring sudden acute respiratory syndrome coronavirus 2 (SARS-CoV-2) spike (CoV S) glycoprotein of SEQ ID NO: 2 and an adjuvant. The variants of the SARS-CoV-2 mutation include Alpha (B.1.1.7), Beta (B.1.351), Gamma (P.1), Delta (B.1.617.2) and Mu (B.1.621) variant; or a combination of variants thereof, including both identified and unidentified variants thereof. In one variation, the above method suppresses infection and prevents severe respiratory distress syndrome cause by SARS-CoV-2 or a SARS-CoV-2 variant thereof.

In another aspect of the above method, the modified spike protein is encoded by a gene sequence of SEQ ID NO: 1. In one variation, the gene sequence is a polynucleotide comprising of or consisting of SEQ ID NO: 1. In another aspect of the method, the modified spike protein is cloned with a pCNeoMEM vector and expressed by a host cell line CHO-DG44. In another aspect of the above method, the SARS-CoV-2 variant is Delta (B.1.617.2). In yet another aspect of the method, the composition comprising a modified spike (S) protein comprising of SEQ ID NO: 2; and an adjuvant. In yet another aspect of the method, the modified spike protein is cloned with a pCNeoMEM vector and expressed by a host cell line CHO-DG44. In another aspect of the method, the dose comprising the modified spike protein ranges from 5 μg/mL to 100 μg/mL.

In one variation, the concentration of the modified spike protein in the composition ranges from 3 μg/mL to 100 μg/mL, about 3 μg/mL to 75 μg/mL, about 3 μg/mL to 50 μg/mL, about 3 μg/mL to 25 μg/mL or about 3 μg/mL to 15 μg/mL. In another variation, the concentration of the modified spike protein ranges from 1 μg/mL to 300 μg/mL. In another variation, the composition comprises about 3 μg, 5 μg, 7 μg, 9 μg, 10 μg, 15 μg, 20 μg, 30 μg, 40 μg or about 50 μg of the modified spike protein. In another variation, the composition comprises from about 3 μg to about 50 μg of the modified spike protein.

In yet another aspect of the method, the dose comprising the modified spike protein is 3 μg/mL or 5 μg/mL. In another aspect of the method, the subject is administered a first dose at day 0 and a boost or second dose at day 21; or a boost or second dose at day 30. In one variation of the method, a single dose of the composition is administered. In another variation of the above methods, the second dose is administered within about 30 days after the first composition is administered. In another variation of the above method, the first and second compositions are the same. In another variation of the method, the first composition is different from the second composition, in terms of concentration, dose and the amount and type of adjuvant present, as disclosed herein.

In another aspect of the method, the first dose composition is selected from the group consisting of the modified spike protein of SEQ ID NO: 2, a plasmid DNA encoding the modified spike protein of SEQ ID NO: 2, a viral vector encoding the modified spike protein of SEQ ID NO: 2 and an inactivated modified spike protein of SEQ ID NO: 2. In yet another aspect of the method, the second dose composition is selected from the group consisting of the modified spike protein of SEQ ID NO: 2, a plasmid DNA encoding the modified spike protein of SEQ ID NO: 2, a viral vector encoding the modified spike protein of SEQ ID NO: 2 and an inactivated modified spike protein of SEQ ID NO: 2.

The foregoing examples of the related art and limitations are intended to be illustrative and not exclusive. Other limitations of the related art will become apparent to those of skill in the art upon a reading of the specification and a study of the drawings or figures as provided herein. In addition to the exemplary embodiments, aspects and variations described above, further embodiments, aspects and variations will become apparent by reference to the drawings and figures and by examination of the following descriptions.

### DETAILED DESCRIPTION OF THE INVENTION

### Definitions:

Unless specifically noted otherwise herein, the definitions of the terms used are standard definitions used in the art of amino acids, peptide and nucleic acid synthesis, vaccine development, immunology and pharmaceutical sciences. Exemplary embodiments, aspects and variations are illustratived in the figures and drawings, and it is intended that the embodiments, aspects and variations, and the figures and drawings disclosed herein are to be considered illustrative and not limiting.

The terms “immunogen,” “antigen,” and “epitope” refer to substances such as proteins, including glycoproteins and peptides, that are capable of eliciting an immune response.

An “immunogenic composition” is a composition that comprises an antigen where administration of the composition to a subject results in the development in the subject of a humoral and/or a cellular immune response to the antigen.

The terms “treat,” “treatment” and “treating” refer to an approach for obtaining beneficial or desired results, for example, clinical results. As used herein, beneficial or desired results may include inhibiting or suppressing the initiation or progression of an infection or a disease; ameliorating or reducing the development of, symptoms of an infection or disease; or a combination thereof.

The term “prevention” is used interchangeably with “prophylaxis” and may mean complete prevention of an infection or disease, or prevention of the development of symptoms of that infection or disease; a delay in the onset of an infection or disease or its symptoms; or a decrease in the severity of a subsequently developed infection or disease or its symptoms.

An “effective dose” or “effective amount” refers to an amount of an immunogen sufficient to induce an immune response that reduces at least one symptom of pathogen infection. An effective dose or effective amount may be determined for example, by measuring amounts of neutralizing secretory and/or serum antibodies, e.g., by plaque neutralization, complement fixation, enzyme-linked immunosorbent (ELISA), microneutralization assay, and as described herein.

The term “vaccine” refers to an immunogenic composition, such as an immunogen derived from a pathogen, which is used to induce an immune response against the pathogen that provides protective immunity (e g , immunity that protects a subject against infection with the pathogen and/or reduces the severity of the disease or condition caused by infection with the pathogen). The protective immune response may include formation of antibodies and/or a cell-mediated response. The term “vaccine” may also refer to a suspension or solution of an immunogen that is administered to a subject to produce protective immunity.

The term “subject” includes humans and other animals. Typically, the subject is a human For example, the subject may be an adult, a teenager, a child (2 years to 14 years of age), an infant (birth to 2 year), or a neonate (up to 2 months). In particular aspects, the subject is up to 4 months old, or up to 6 months old. In some aspects, the adults are seniors about 65 years or older, or about 60 years or older. In some aspects, the subject is a pregnant woman or a woman intending to become pregnant. In other aspects, subject is not a human; such as a non-human primate; for example, a baboon, a chimpanzee, a gorilla or a macaque. In certain aspects, the subject may be a pet, such as a dog or cat.

The term “pharmaceutically acceptable” means being approved by a regulatory agency of a U.S. Federal or a state government or listed in the U.S. Pharmacopeia, European Pharmacopeia or other generally recognized pharmacopeia for use in mammals, and more particularly in humans. These compositions can be useful as a vaccine and/or antigenic compositions for inducing a protective immune response in a vertebrate.

“Pharmaceutically acceptable salts” means salt compositions that is generally considered to have the desired pharmacological activity, is considered to be safe, non-toxic and is acceptable for veterinary and human pharmaceutical applications. Such salts include acid addition salts formed with inorganic acids such as hydrochloric acid, hydrobromic acid, sulfuric acid, phosphoric acid, and the like; or with organic acids such as acetic acid, propionic acid, hexanoic acid, malonic acid, succinic acid, malic acid, citric acid, gluconic acid, salicylic acid and the like.

“Therapeutically effective amount” means a drug or a composition amount that elicits any of the biological effects listed in the specification.

Disclosed herein is a Covid-19 subunit vaccine, referred to herein as Nanocovax. The vaccine is based on recombinant protein technology to produce the secreted form of the S protein of SARS-CoV-2. In one aspect, the secreted Spike protein is soluble in the applicable medium. A gene encoding the S protein was constructed with the wild-type sequence of the full-length S protein. The construct was transfected into CHO cells, named CHO-spike cells, with the highest S protein expression selected. The S protein of SARS-CoV-2 was then absorbed into an adjuvant, such as aluminum hydroxide gel adjuvant (Alhydrogel®; Croda, Denmark). Nanocovax is a recombinant severe acute respiratory syndrome coronavirus 2 subunit vaccine composed of full-length prefusion stabilized recombinant SARS-CoV-2 spike glycoproteins (S-2P) and an adjuvant, as disclosed herein.

### Plasmid Construction, Cell Clone and Purification:

The pCNeoMEM vector was used as the expression vector. The pCNeoMEM plasmid contains a G418 resistance gene used as a selection marker, a MoMLV promoter to express the target gene, a human universal chromatin opening element (UCOE), untranslated regions from the Chinese hamster EEF1A1 gene (eEF1A1), and a synthetic matrix attachment sequence (sMAR). The gene encoding the Spike protein of SARS-CoV-2 (UniProt PODTC2) codon-optimized for expression in CHO cells was synthesized by Genscript (Piscataway, N.J., USA). The optimized DNA fragment was cloned in the expression vector to create pCNeoMEM-S, which was completely sequenced by NextGen technology at the Center for Computational and Integrative Biology, Harvard University.

### Cell Culture and Protein Expression

CHO cells (cGMP bank, Thermo Fisher Scientific, Waltham, Mass., USA) were propagated and maintained in the animal component free, chemically defined medium, PowerCHO-2, (Lonza, Walkersville, Md., USA) in a 37° C. and 5% CO₂. Suspension cells were routinely subcultured every 2-3 days at a cell concentration of 2×10⁵ cells/mL. Before transfection, the cells were seeded at 1×10⁶ cells/mL into a 6-well plate and cultured for 24 h. For transfection, Lipofectamine LTX Reagent (Thermo Fisher Scientific, Waltham, Mass., USA) was used following manufacturer's instructions. In optimized conditions, a total of 3 μg of plasmids was used to transfect into 1×10⁶ cells per well in 6-well plates using PowerCHO-2 medium. After 48 h of transfection, the expression of S protein was evaluated by using ELISA. After transient transfection, selection was performed by culturing transfected cells in PowerCHO-2 medium, supplemented with 400 μg/mL of Geneticin G418 (Sigma-Aldrich, St. Louis, Mo., USA). The selective medium was replaced every 2-3 days for 3 weeks to obtain stable cell lines. Single-cell cloning was then initiated by limiting dilution. The clones with higher productivity were selected to create master cell bank (MCB) and working cell bank (WCB). Process development was performed using the high throughput multi-parallel Bioreactor Ambr 15 and Ambr 250 (Sartorius Stedim Biotech, Gottingen, Germany) Protein production at pilot scale for nonclinical trial material was archived in 500 L Bioreactor (BIOSTAT STR®500, Sartorius Stedim Biotech, Gottingen, Germany) using a fed-batch process using PowerCHO-2 medium as basal medium, supplement with Feed 3 (Excellgene, Monthey, Switzerland), trace elements and activated sugars (n=3).

### Protein Purification

The purification of S protein from cell broth was performed using on AKTA Pilot 600R system (GE Healthcare, Little Chalfont, UK). After harvesting, the cell supernatant was clarified by depth-filtration (Merck Millipore, Darmstadt, Germany) to remove cells and purified by consecutive chromatography steps. First, sample was loaded onto Blue Sepharose column (Cytiva, Marlborough, Mass., USA) to specifically collect S proteins and then treated at low pH 3.2-3.5 for 60-70 min to inactivate virus. Next, sample was flowed through Q membrane, and then Phenyl membrane (Sartorius Stedim Biotech, Gottingen, Germany) to remove host cell DNA, proteins and endotoxin. Exotic viruses were removed by nano-filtration. Purified S proteins were exchanged to storage buffer and then filtered through 0.22 μm filter (Sartorius Stedim Biotech, Gottingen, Germany). The concentration of purified protein was determined by ELISA. The purity of protein was determined by SE-HPLC, residual host cell DNA assay, and residual host cell protein assay.

### Spike Protein of SARS-CoV-2 Analysis

### Spike Protein Identification

### SDS-PAGE and Western Blot Assay

Purified proteins were mixed with loading buffer, and loaded onto SDS-PAGE gels. Proteins were electrophoresed for 110 minutes at 80V, 500 mA and visualized using Coomassie Brilliant Blue G250 staining (Sigma-Aldrich, St. Louis, Mo., USA) or transferred to a nitro cellulose blotting membrane (Sigma-Aldrich, St. Louis, Mo., USA) at a constant current of 250 mA, for 90 minutes. The membranes were blocked in 0.5% BSA (Sigma-Aldrich, St. Louis, Mo., USA) and incubated with human anti-S1 antibody (Abcam, Cambridge, Mass., UK) for 3 hours at room temperature. After washing, the membranes were incubated with horseradish peroxidase (HRP)-linked rabbit anti human IgG antibody (Abcam, Cambridge, Mass., UK) for 1 hour at room temperature. Membranes were washed 3 times with PBST and the protein bands were visualized by enhancing TMB substrate solution (Sigma-Aldrich, St. Louis, Mo., USA).

### ELISA Assay

The culture supernatant sample or purified S protein was coated on each well of the microtiter plate overnight at 4° C. After blocking the wells with 1% BSA for 1 hour, dilutions of human anti-S1 antibody (Abcam, Cambridge, Mass., UK) were added to wells and incubated for 3 hours at room temperature. After washing, horseradish peroxidase (HRP)-conjugated rabbit anti human IgG antibody (Abcam, Cambridge, Mass., UK) was added and incubated for 1 hour at room temperature. Bound antibodies were detected using TMB substrate (Sigma-Aldrich, St. Louis, Mo., USA). Absorbance was read at 450 nm on Multimode Plate Reader (Promega, Madison, Wis., USA).

### Molecular Mass Assay

The molecular mass of purified protein was measured by MALDI-TOF-MS at Biofidus AG Company. Recombinant SARS-COV-2 Spike protein was desalted and concentrated with C4 ZipTips (Merck Millipore, Darmstadt, Germany) and spotted on a ground steel target using 2′,5′-Dihydroxyacetophenone. The sample was measured via MALDI-TOF-MS (UltrafleXtreme, Bruker Daltonik GmbH, Bremen Germany) in positive ion mode. Recorded MS spectra were processed by the software FlexAnalysis (Bruker Daltonik GmbH, Bremen Germany).

### Peptide Mapping Assay

Peptide mapping of purified protein was performed by Biofidus AG Company. The peptide mapping of a recombinant SARS-CoV-2 spike protein was measured by LC-ESI-MS using different digestion strategies. The focus of the peptide mapping was sequence verification and analysis of N- and C-terminal modifications. Mass spectrometric analysis was performed with a Compact QTOF mass spectrometer (Bruker Daltonik GmbH, Bremen Germany) The recorded LC-ESI-MS and -MS/MS spectra were processed, annotated and searched against a customized sequence database using Mascot (Matrix Science). Modified peptides were identified by their exact mass and retention time and quantified by their mass spectrometric signal intensity.

### In Vivo Immunigenicity Evaluation of Nanocovax Vaccine in Balb/c Mice, Syrian Hamsters, and Non-Human Primate Models

### Animal Vaccination

Balb/c mice (6-10 weeks old), syrian hamsters (Mesocricetus auratus: 8-12 weeks old), and northern pig-tailed macaques (Macaca leonina: 4-5 years old) were used for immunological study. They were immunized intramuscularly with Nanocovax at doses of 25 μg, 50 μg, 75 μg and 100 μg, their serum samples were collected quantification of spike protein specific IgG antibodies by ELISA. Blood samples were collected and let to clot at room temperature for 60 min, then centrifuged at 1000×g for 15 min. Upper serum fraction was collected and heat-inactivated at 56° C. in 30 minutes before use or kept at −20° C.

### In Vitro Surrogate Virus Neutralization Assay

The virus neutralization ability of antibodies in sera of Balb/C mice, hamster and non-human private were determined by the virus surrogate neutralization kit (cat #L00847, Genscript, Singapore). The percent of neutralizing virus in sera were determined according to the manufacturer's protocol.

### In Vitro SARS-CoV-2 Virus Neutralization Assay (PRNT)

The PRNT assay detects and quantifies neutralizing antibody titer for SARS-CoV-2 in serum samples. Sera were 2-fold serially diluted in culture medium with a starting dilution of either 1:10 or 1:20. The diluted sera were mixed with 100 plaque-forming units (PFU) of SARS-CoV-2 virus for 1 h at 37° C. The virus—serum mixtures were added onto Vero E6 cell monolayers and incubated 1 h at 37° C. in 5% CO2 incubator. Then the plates were overlaid with 1% agarose in cell culture medium and incubated for 4 days when the plates were fixed and stained. Antibody titres were defined as the highest serum dilution that resulted in >50% (PRNT₅₀) reduction in the number of plaques. PRNT was performed in duplicate using 24-well tissue culture plates in a biosafety level 3 facility (BSL3) in the National Institute of Hygiene and Epidemiology (NIHE), Hanoi, Vietnam, as adapted from Okba N. et al, Severe Acute Respiratory Syndrome Coronavirus 2-Specific Antibody Responses in Coronavirus Disease Patients. Emerging Infectious Diseases 2020; 26:1478-88. https://doi.org/10.3201/eid2607.200841.

### Protective Efficacy Evaluation of Nanocovax Vaccine in Syrian Hamsters Viral Challenge Study

The hamsters were assigned to the following groups: (1) vaccinated with Nanocovax on day 0 and day 7 and then challenged with the high level of the SARS-CoV-2 virus on day 14 by the intranasal route (TCID₅₀=2×10⁵); (2) vaccinated with Nanocovax on day 0 and day 7 and then challenged with the low level of the SARS-CoV-2 virus on day 14 by the intranasal route (TCID₅₀=1×10³), and (3) placebo injection with PBS and challenged with the high/low level of the SARS-CoV-2 virus on day 14 by the intranasal route (TCID₅₀=2×10⁵ and 1×10³). Baseline body weights were measured before infection. Animals were monitored for signs of morbidity (such as weight loss, ruffled hair, sweating, etc.) for 14 days. On day 28, their lungs were collected for detection of SARS-CoV-2 by Real-time RT-PCR. Quantitative detection of SARS-CoV-2 on the lung samples was adapted from WHO's protocol. See Corman V. et al. Diagnostic detection of 2019-nCoV by real-time RT-RCR. Carité Berlin 2020:1-13. Infection doses were chosen to base on Imai et al. Syrian hamsters as a small animal model for SARS-CoV-2 infection and countermeasure development. Proceedings of the National Academy of Sciences of the United States of America 2020; 117:16587-95. https://doi.org/10.1073/pnas.2009799117.

### Real-Time RT-PCR

Real-time RT-PCR was performed to quantify SARS-CoV-2. This PCR amplifies the envelope (E) gene of SARS-CoV-2 using forward primer 5′-ACAGGTACGTTA-ATAGTTAATAGC-3′; reverse primer: 5′-ATATTGCAGCAGTACGCACAC-3′; and probe: 5′-FAM-ACACTAGCCATCCTTACTGCGCTTCGBBQ-3′. The Real-time RT-PCR assays were conducted using TagMan One-Step RT-PCR kit (Thermo Fisher Scientific, Waltham, Mass., USA) on Real-Time PCR System (Biorad, Calif., USA) with the following cycling conditions: 55° C. for 10 min for reverse transcription, 95° C. for 3 min, and 45 cycles of 95° C. for 15 s and 58° C. for 30 s. The absolute copy number of viral loads was determined using serially diluted DNA control targeting the E gene of SARS-CoV-2.

### Safety Evaluation of Nanocovax Vaccine

According to ICH/GLP guidelines with minor modification, single-dose and repeat-dose toxicity studies were performed on adult male and female mice and rats with a few modifications. The animals were examined and weighed prior to starting the designed experiment. In single-dose toxicity test, a total of 60 mice of both sexes were divided into 6 groups (n=10, 5 females and 5 males) and intramuscular injected (I.M) Nanocovax at single doses of 25 μg, 50 μg, 75 μg and 100 μg or placebo. Untreated mice were used as a control. All animals were regularly monitored continuously within the first 4 hours for behavioural and pathological signs and then daily for the next 14 days for mortality, abnormal behaviour and body weight. In repeat-dose toxicity test, a total of 36 rats were divided into 6 groups (n=6; 3 males and 3 females). Rats were intramuscular injected (I.M) Nanocovax daily doses of 25 μg, 50 μg, 75 μg and 100 μg or placebo for 28 days. The mortality and clinical signs were observed daily and the body weight was determined at the indicated time points during the experimental period. At the end of treatment, all tested rats were anesthetized to collect blood samples for analysis of biochemical and hematological parameters. The kidneys, spleen and liver were immediately isolated, weighed individually and examined histologically. This study was carried out in strict adherence to the animal laboratory of Nanogen pharmaceutical Company, the National Institute of drug quality control (NIDQC), and the laboratory of Hanoi Medical University (HMU). The processes were designed according to the guide of ICH/GCP, Drug administration of Vietnam, as well ACTD and approved by Ethics committee of Ministry of Health.

### Statistical Analysis

The collected data were statistically analyzed using Grapthpad Prism, version 5 (Grapthpad Software). Data are expressed as mean±standard deviation (SD). Statistical analysis was performed using two-way ANOVA analysis with Bonferroni post-tests and one-way ANOVA followed by the Newman-Keuls multiple comparison test to assess the difference between the various groups. Differences described as significant in the text correspond to *p<0.05; **p<0.01; ***p<0.001.

### Recombinant SAR-CoV-2 Spike Protein Production

To generate SAR-CoV-2 antigen for vaccine development, we designed an optimized DNA sequence encoding the extracellular domain sequence of the Spike protein which has some changes in (1) the S1/S2 Furin cleavage site to minimize the cleavage of S1/S2 during protein production, (2) the two Proline in S2 domain (986-987) to enhance Prefusion-stabilized SARS-CoV-2 Spikes and (3) the 9-arginine residue in the C-terminus. The recombinant SAR-CoV-2 Spike proteins (S protein)-producing clone was selected on the basis of phenotypic stability, productivity and key quality properties of the desired product, named CHO-Spike cells. Nucleotide sequence of the gene integrated in the CHO-Spike cell genome was confirmed by sequencing of complementary DNAs.

Upstream production of S protein by CHO-Spike cells was performed in a 500 L Bioreactor using a fed-batch process with Feed 3 supplement. The cells were cultured in 380 L PowerCHO-2 medium at initial concentration of 0.5×10⁶ cells/mL, and fed with 1.5% of final volume of Feed 3 daily from day 3 to day 13. The temperature was initiated at 37° C. and shifted to 32° C. at day 3 of cell culture. Cell density, cell viability and protein titer were determined at indicated time. The CHO-Spike cells could obtain a maximum cell density of 9.48±0.13×10⁶ cell/mL and survive for 13 days with the cell viability of 80%. At the end of cultivation, the culture supernatant was harvested and purified for further tests. The data showed that the maximum protein titer was 61.4±1.06 mg/L on the harvest day. The SDS-PAGE of harvest sample indicated that the predicted protein was at about 180 kDa, similar with S protein molecular weight. This protein bound specifically to anti-S1 protein antibody. The harvest sample was purified by conducting on AKTA Pilot 600R. The results of ELISA assay also indicated the purified protein concentration was 21.4±0.18 g per batch with 96.56% of purity. Residual host cell protein and DNA were not detected in purified S protein.

### Characterization of Recombinant SARS-CoV-2 Spike Protein

SDS-PAGE analysis showed that the molecular weight of purified S protein is about 180 kDa. Consistent with SDS-PAGE results, anti-S1 antibodies bound specifically to predicted S protein as assayed by Western Blot analysis. The data of intact mass analysis by MALDI-MS also confirmed that the S protein mass was determined to be 185668±1849 Da. The N- and C-terminal sequence and peptide mapping of recombinant S protein suggested a truncation of N-terminal serine and complete pyroglutamate formation of the N-terminal glutamine The C-terminus shows a high heterogeneity with a C-terminal peptide with truncation of AA 1215-1222 as the most abundant variant. The conservation of recombinant S protein was evaluated by comparing N- and C-terminal peptide sequences to complete nonredundant database of protein sequences storage at NCBI, using the BLASTP computer program. Data showed that N- and C-terminal peptide sequences matched 100% to published SARS-CoV-2 Spike protein sequences.

### Immunogenicity of Nanocovax Vaccine in Animal Models

Balb/c mice were injected with various dosages (25 μg, 50 μg, 75 μg and 100 μg) of vaccine absorbed with 0.5 mg A1³⁺ (aluminum hydroxide adjuvant) for two times with 7-days. The levels of total specific IgG were determined by ELISA on day 14 post-priming injection. The result shows that IgG levels significantly increased in a dose-dependent manner Syrian hamsters were vaccinated with various doses of Nanocovax (25 μg, 50 μg, 75 μg and 100 μg). The antibodies were detected on day 28 and day 45 post-priming. Data shows that the amount of S protein-specific IgG in vaccinated hamster groups on D28 were 171.5-fold, 219.8-fold, 222.6-fold and 253.0-fold compared with the placebo group, respectively. The level of antibodies was decreased on day 45 in the experiments and respective 144.0-fold, 193.8-fold, 240.5-fold and 196.6-fold compared with the placebo group.

To confirm the immunogenicity of Nanocovax vaccine, northern pig-tailed macaques were studied. Monkeys were administered 2 doses by I.M injection with various concentrations of Nanocovax (25 μg, 50 μg, 75 μg and 100 μg), or PBS as a negative control. After boosting injection on day 7, the blood was collected on day 14, day 28 and day 45 to detect the level of antibodies. The levels of IgG in vaccinated groups were significantly increased in a time-dependent manner On day 28 post-priming injection, the IgG levels were slightly increased in all vaccinated groups and were 39.02-fold, 68.58-fold, 87.82-fold and 97.37-fold, respectively. On day 45, the amount of IgG in the serum in all vaccinated groups were significantly increased compared with the control group. The IgG levels of vaccinated monkeys were 126.5-fold, 129.1-fold, 159.95-fold and 205.12-fold, respectively.

Sera were collected from vaccinated animal models and the percent of neutralizing antibodies was measured by a surrogate virus neutralization test (SARS-CoV-2 sVNT Kit). For the Balb/C mice model, the blood was collected from the mice after immunization on day 14 to detect the percent of inhibition. Four dosages of Nanocovax (25 μg, 50 μg, 75 μg and 100 μg) strongly inhibited SARS-CoV-2 virus compared with PBS group (***P<0.001). The inhibition by Nanocovax 50 μg was not significantly different from Nanocovax 75 μg (89.60% versus 89.40%). A significant difference was observed between the 25 μg group compared with the 50 μg group (77.5% versus 89.60%), as well as the 75 μg group versus the 100 μg group (89.40% versus 97.30%). For the syrian hamster model, blood was collected on day 28 and day 45 post-priming injection with four dosages of Nanocovax. The percent of inhibition was detected by SARS-CoV-2 sVNT Kit. The results indicated that no significant difference (P>0.05) was observed among all groups on day 28 and day 45 post-priming injection with four dosages of Nanocovax vaccine (25 μg, 50 μg, 75 μg and 100 μg) respectively. Vaccinated hamster groups showed significant inhibition by antibodies in sera compared with the non-vaccinated hamster group (***P<0.001).

We next measured the virus neutralization ability of antibodies in the non-human primate model. The monkeys were immunized twice a week with Nanocovax with dosages of 25 μg, 50 μg, 75 μg and 100 μg. Sera were collected on day 14, day 28 and day 45 after priming I.M injection to determine inhibition of S protein using the virus surrogate neutralization KIT. The monkeys vaccinated with the 25 μg dosage of Nanocovax produced no significant inhibition on day 14 (the percent of inhibition was 26.14%, lower than the 30% Cutoff value). On day 28 and day 45, the monkey groups immunized with four dosages of Nanocovax had positive results (>30% Cutoff value) and no significant difference in the percent of inhibition.

SARS-CoV-2-neutralizing antibodies can be measured by the PRNT₅₀ viral neutralization assay, which is used to determine the levels of neutralizing antibodies in sera from the vaccinated animals. The specimens of Balb/C immunized with Nanocovax 25 μg, 50 μg, 75 μg and 100 μg had respective SARS-CoV-2-neutralizing antibodies (1/PRNT₅₀) titer from 40 to 640. All specimens of hamsters received Nanocovax 25 μg, 50 μg, 75 μg and 100 μg had respective SARS-CoV-2-neutralizing (1/PRNT₅₀) titer from 20 to 320.

Protective efficacy of Nanocovax vaccine in hamster model

After challenged with the high or low level of the SARS-CoV-2 virus, three vaccinated hamsters from each group had no signs of weight loss: weight maintenance for 1-2 days post-infection and weight gain continued from day 3 post-infection and day 14 post-infection (11.8% to 14.5%). No symptoms were observed in vaccinated animals: no shortness of breath, ruffled fur or lethargy were observed in any vaccinated hamsters receiving the low and high doses of SARS-CoV-2. Three control hamsters exhibited ruffled fur, lethargy and sweating symptoms on day 1 and 2 after the virus challenge. Two of three animals showed severe weight loss by day 7 or 8 post-infection (13.2% to 16.4%), and gained weight slowly from day 8 or 9 after the challenge test.

On day 28, the lungs from the vaccinated hamster group and the non-vaccinated group were collected to detect SARS-CoV-2 by Realtime RT-PCR. The results showed that no SARS-CoV-2 virus-specific RNA was detected in lung samples of the vaccinated group after 14 days of challenge with viral concentrations 2×10⁵ TCID₅₀ and 1×10³ TCID₅₀ compared with the non-vaccinated group (Ct=30.33 and Ct=31.22, respectively).

### Safety Evaluation of Nanocovax Vaccine

In the single-dose toxicity analysis, data showed no mortality and no drug-related toxicity signs in the tested mice at all tested doses within 14 days. There were no significant differences between groups for body weight gain. Macroscopic examination showed no abnormalities in physical appearance of liver, heart, kidneys, spleen, lungs and intestines between the treated groups and the control group. In the repeat-dose toxicity analysis, the treated rats and the controls appeared uniformly healthy and no lethality was recorded in tested rats during the 28-day treatment period. There were no clinically abnormal symptoms in general behaviour between treatment and control groups. In comparison with control rats, the body weight of female and male rats gradually increased during the test period. There was no statistically significant difference in body weight gain between the treated groups and the control group. Similarly, no significant difference was observed in organ weights of rats, both males and females between treated groups and control group. The indicators of the hematological parameters and biochemical parameters of rats had no significant difference in vaccinated groups and control groups. Macroscopic observations showed that there were no lesions and no abnormalities of physical appearance of liver, kidneys and spleen observed in all groups. No significant macroscopic changes were observed between groups. The results from histopathological analysis revealed that none of kidney, liver and spleen tissues of both male and female rats that received Nanocovax daily at all doses showed any damage or pathological alterations under microscopic observation. The results indicated that both single-dose and repeat-dose toxicity studies of Nanocovax indicated the safety of Nanocovax at all tested doses.

### Vaccine Development

The development of vaccines with high immunogenicity and safety are the major concerns for control of the global COVID-19 pandemic and protection of further illness and fatalities. Vaccine candidates are currently under development using different platforms, such as inactivated vaccines, live-attenuated vaccines, viral vector (adenovirus) vaccines, DNA vaccines and mRNA vaccines. In other platforms, recombinant protein vaccines were used together with adjuvants to enhance adaptive immunity. Aluminum salt-based adjuvants are approved for many human vaccines such as Hepatitis A, Hepatitis B, Haemophilus influenza type b (Hib) and Diphtheria-tetanus-pertusis (DtaP, Tdap). See Tomljenovic L et al. Aluminum Vaccine Adjuvants: Are they Safe? Current Medicinal Chemistry 2011; 18:2630-7. https://doi.org/10.2174/092986711795933740. Aluminum adjuvants have been applied in SARS-CoV-2 vaccines such as RBD vaccine; spike protein vaccine; inactivated virus vaccine; VLP vaccine. Gupta T et al. Potential adjuvants for the development of a SARS-CoV-2 vaccine based on experimental results from similar coronaviruses. International Immunopharmacology 2020; 86. https://doi.org/10.1016/j.intimp.2020.106717.

The Nanocovax vaccine, which is a spike protein, was designed and harvested at final concentration (21.06 g per batch) after purification with 96.56% purity. The spike protein was then formulated with aluminum hydroxide adjuvant.

To evaluate the immunogenicity of Nanocovax vaccine, three animal models including Balb/C mice; Syrian hamster mice, and non-human primates were used. The antibody IgG titer in sera from vaccinated Balb/C groups with various concentrations of Nanocovax (25 μg, 50 μg, 75 μg and 100 μg) significantly increased in a dose-dependent manner on D14. The levels of IgG titer were similar on D28, as well D45 by using the hamster model. In addition, the amount of IgG in serum of monkeys in all vaccinated groups was significantly in a time-dependent manner

The neutralizing antibodies to SARS-CoV-2 are needed to determine not only the infection rate, immunity, but also vaccine efficacy during clinical trials. Tan CW et al. A SARS-CoV-2 surrogate virus neutralization test based on antibody-mediated blockage of ACE2-spike protein-protein interaction. Nature Biotechnology 2020; 38:1073-8. https://doi.org/10.1038/s41587-020-0631-z. Sera were collected from vaccinated animal models, as well non-human primates to measure the percent of neutralizing antibodies by surrogate virus neutralization test. The antibody in sera can neutralize SARS-CoV-2 virus with a high percent of inhibition. In agreement with the result from Bošnjak, a strong positive correlation between surrogate virus neutralization test and the levels of S-specific IgG. Bošnjak et al. Low serum neutralizing anti-SARS-CoV-2 S antibody levels in mildly affected COVID-19 convalescent patients revealed by two different detection methods. Cellular and Molecular Immunology 2021; 18:936-44. http://doi.org/10.1038/s41423-020-00573.9. Our animal models showed that the IgG levels in sera from vaccinated animals with Nanocovax increased high affinity viral neutralizing antibodies. SARS-CoV-2-neutralizing antibodies can be measure by PRNT₅₀. Sera from Balb/C and Hamster models had neutralizing antibodies titer from 80 to 640, and 20 from 320, respectively.

The efficacy of Nanocovax vaccine on hamster was used in a study. After challenging with SARS-CoV-2 virus, no symptoms were observed such as shortness of breath, ruffled fur, or lethargy as well as weight loss in all vaccinated mice verse control hamster groups. On day 28, SARS-CoV-2 virus-specific RNA was detected in the lung samples of non-vaccinated groups by Real-time RT-PCR.

The safety of the Nanocovax vaccine was investigated based on single-dose and repeat-dose toxicity studies. The results showed that Nanocovax vaccine has no single-dose and repeated-dose toxicity effect testing on mice (Mus musculus val. Albino), and Rat (Rattus norvegicus) with four dosages (25 μg, 50 μg, 75 μg and 100 μg). Nanocovax vaccine demonstrated immunogenicity and safety based on animal models as well as non-human primate model. Trial vaccine, adjuvant and placebo:

The recombinant SARS-CoV-2 spike (S) glycoprotein in Nanocovax were constructed with 682-QQAQ-685 mutations for protease resistance, and two proline substitutions (K986P and V987P) for stabilized prefusion conformation. The production of the full-length (including the transmembrane domain) recombinant S protein was optimized in the established Chinese Hamster Ovary (CHO) cell-expression system. Clinical grade aluminum hydroxide was manufactured by Croda (Denmark). Recombinant SARS-CoV-2 S protein were absorbed to aluminum adjuvant in mild shaking condition for 18 hours at 2° C. to 8° C. Placebo was sterile 0.05% aluminum.

General Results from Phase 1 and Phase 2 Clinical Trials:

Phase 1 and 2 trials were performed to evaluate the safety and immunogenicity of 25 μg, 50 μg and 75 μg dose strengths of recombinant SARS-CoV-2 S glycoprotein with aluminum adjuvant (0.5 mg/dose) in healthy adults of at least 18 years of age. We conducted a dose-escalation, open label trial (phase 1) and a randomized, double-blind, placebo-controlled trial (phase 2) to evaluate the safety and immunogenicity of the Nanocovax vaccine in 25 microgram (mcg or μg), 50 μg and 75 μg doses, aluminum hydroxide adjuvanted. In phase 1, 60 participants received two intramuscular injection of the vaccine following dose-escalation procedure. The primary outcomes were reactogenicity and laboratory tests to evaluate the vaccine safety. Phase 2, which included 560 healthy adults, the primary outcomes are vaccine safety and anti-S IgG antibody response. Secondary outcomes were surrogate virus neutralization, wild-type SARS-CoV-2 neutralization and T-cell responses by intracellular staining (ICS) for interferon gamma (IFNgγ). We performed primary analyses at day 35 and day 42.

Phase 1 Summary No serious adverse events (SAE) were observed for all 60 participants. Most adverse events (AE) were grade 1 and disappeared shortly after injection. For phase 2 study, after randomization, 480 participants were assigned to receive the vaccine with adjuvant, and 80 participants were assigned to receive placebo. Reactogenicity was absent or mild in the majority of participants and of short duration (mean, ≤3 days). Unsolicited adverse events were mild in most participants. There were no serious adverse events related to Nanocovax. Nanocovax induced robust anti-S antibody responses. There was no statistical difference in antibody responses among dose strengths on Day 42, in terms of anti S-IgG level and neutralizing antibody titer. Up to 42 days, Nanocovax vaccine was safe, well tolerated and induced robust immune responses. Nanocovax at 25 μg may be used for Phase 3 to evaluate the vaccine efficacy. (Nanogen Pharmaceutical Biotechnology JSC; ClinicalTrials.gov number, NCT04683484.)

### Trial Design and Oversight

Phase 1 trial was conducted as an open-labeled, dose-escalation study with the emphasis on the vaccine safety. Eligible participants were healthy men and nonpregnant women, 18 to 50 years of age with body-mass index (BMI) of 18 to 27 kg/m². 60 participants were allocated into 2:2:2 ratio of 25 μg, 50 μg and 75 μg dose groups, respectively. The first 3 participants of 25 μg dose group were vaccinated and monitored for 72 hours. After no SAE were observed, all remaining participants in this group plus 3 participants in 50 μg dose group would be vaccinated and monitored for 72 hours. If no SAE were observed among 3 participants of 50 dose group, the remaining participant in this group plus 3 participants in 75 μg dose group would be vaccinated. If no SAE were observed among 3 participants in 75 μg dose group, the remaining participants in this group will be vaccinated. All participants received 2 intramuscular injections of the vaccine into the deltoid on day 0 and day 28. Sample size of phase 1 was not based on formal statistical power calculation but on the range of 30-150.

Phase 2 trial was conducted at two sites. This was a randomized, double-blind, placebo-controlled study. Eligible participants were healthy men and nonpregnant women, at least 18 years of age with BMI of 17 to 35. They were stratified into 3 age groups: from full 18 to full 45 years old, from 46 to 60 years old, and over 60 years old. The sample size of phase 2 was calculated based on the estimated probability of observing an adverse event. A total of 560 participants were randomly assigned to 4 groups, into 2:2:2:1 ratio for 25 μg, 50 μg, 75 μg and placebo, respectively. 480 volunteers received the vaccine (160 volunteers receiving 25 μg dose; 160 volunteers receiving 50 μg dose and 160 volunteers receiving dose of 75 μg dose) and 80 volunteers receive the placebo (aluminum adjuvant only). All participants received 2 intramuscular injections of the vaccine or the placebo into the deltoid on day 0 and day 28. Trial staffs responsible for the vaccine preparation and administration, and participants were unaware of vaccine assignment. Randomization lists, using block randomization stratified by study group and study site, were generated. Computer randomization was done with full allocation concealment within the secure web platform used for the study electronic case report form (provided by Medprove company).

All participants were screened by their medical history, clinical and biological examinations, sampling and laboratory tests (complete blood count, biochemistry, urine analysis, testing pregnancy and diagnostic imaging). Participants with a history of Covid-19 or positive results for SARS-CoV-2 at screening period confirmed by real-time reverse transcriptase polymerase-chain-reaction (RT-PCR) were excluded from the trials.

### Safety Assessments

In phase 1, the onsite safety follow-up time after was 72 hours after 1^{st} injection and 24 hours after the 2^{nd} injection. Participants would return to the study site for follow-up visits at scheduled timepoints. In phase 2, the onsite safety follow-up time was 60 minutes after each vaccination. Follow-up visits to evaluate safety were scheduled on days 28, 35, 42, 90 and 180 after vaccination. Participants were observed for 60 minutes after each vaccination for assessment of reactogenicity. In both phases, participants received instruction for self-monitoring and reporting adverse events during 7 days after each vaccination, as facilitated by the use of a diary with predefined reactogenicity. Predefined local (injection site) reactogenicity included pain, tenderness, erythema, and swelling. Predefined systemic reactogenicity included fever, nausea or vomiting, headache, fatigue, malaise, myalgia, and arthralgia.

The primary safety outcomes were the number and percentage of participants with solicited local and systemic adverse events occurred within 7 days after vaccination and laboratory results (serum biochemistry and hematology) at days 0, 7, 28 and 35 according to FDA toxicity scoring. Secondary safety outcomes were occurrence rate and severity rating of unsolicited AE/SAE through day 56 and laboratory results at days 42 and 56 (phase 2 and 1, respectively).

AEs/SAEs were recorded and evaluated based on the Common Terminology Criteria for Adverse Events 5.0 (CTCAE v5.0) and Guidelines for assessing toxicity in healthy volunteers in FDA's Preventive Vaccine Clinical Trial Study. The procedure for recording and evaluating takes place continuously from the time of using the first dose to the end of the last visit in each research volunteer. Adverse events were assessed in terms of severity score (mild, moderate, severe, potentially life-threatening, or fatal), and relatedness to the vaccine. Vital sign measurements were assessed according to FDA toxicity scoring after vaccination. Participants underwent nasopharyngeal swab testing for SARS-CoV-2 on screening day (before 1^{st} vaccination), day 28 (before 2^{nd} vaccination) and any time that they developed symptoms of possible SARS-CoV-2 infection.

### Immunogenicity Assessments

The primary outcome was anti-S IgG responses to Nanocovax evaluated by chemiluminescence immunoassay (CLIA). Secondary outcomes were neutralizing antibody titer evaluated by 50 percent plaque reduction neutralization test (PRNT₅₀) on Wuhan strain and UK variant (B.1.1.7), neutralizing activity evaluated by competitive enzyme-linked immunosorbent assay (ELISA) based surrogate virus neutralization test (sVNT), and T cell response by intracellular cytokine-staining (ICS).

### Statistical Analysis

### Safety Analysis

Local and systemic adverse events occurred during 7 days after each injection were documented (number, percentage) by group (vaccine and placebo) and analyzed to find factors (if any) correlated/associated with the severity of AE. Serious adverse events were classified according to CTCAE 5.0 and statistically described according to the number and percentage of each research group. If an adverse event occurs more than once, analysis is based on only the most severe occurrence and the cause of the event. The results were statistically described as number of and percentage of volunteers having abnormal results or have results changed overtime, by group, compared to baseline values. Values higher than normal ranges/thresholds will be assessed for clinical significance by researchers. AE/SAEs were statistically analyzed the difference in percentage cumulative with 95% confidence interval of the Clopper-Pearson method (Clopper-Pearson 95% CIs) between vaccine dose groups.

### Immune Response Analysis

The geometric anti-S IgG antibody and neutralizing ability of SARS-CoV-2 antibody in the sera at the screening stage is considered as baseline values. Geometric mean concentration (GMC) of anti-S IgG and geometric mean titers (GMT) of neutralizing antibody after vaccination are evaluated at defined time points.

Data were analyzed base on two-sided test with 95% confidence in the t-distribution function: anti-S IgG titer should increase at least 4 times (Clopper-Pearson 95% CIs) as compared to baseline, at defined time points post vaccination, in each dose group.

### Trial Population

Phase 1 trial: 60 participants were allocated into 3 groups of doses: 20 received 25 μg (group 1.1), 20 received 50 μg (group 1.2) and 20 received 75 μg (group 1.3). There was no placebo group and the primary assessment was the vaccine safety.

Phase 2 trial: 560 participants were recruited and randomly assigned into groups of different doses: 161 received 25 μg doses of Nanocovax (group 2.1), 160 received 50 μg doses of Nanocovax (group 2.2), 159 received 75 μg doses of Nanocovax (group 2.3), and 80 received placebo (group 2.4). Participants were stratified into 2 age groups: from over 18 to below 60 years old and over 60 years old. Demographic characteristics of participants in phase 1 and 2.

### Safety Outcomes

In phase 1, no serious adverse events were observed, and vaccination pause rules were not implemented. Overall reactogenicity was largely absent or mild, and second vaccinations were neither withheld nor delayed due to reactogenicity. The percentage of participants in each vaccine group (groups 1.1, 1.2, and 1.3) with solicited adverse events according to the maximum FDA toxicity grade (grade 1: mild, grade 2: moderate, grade 3: severe, grade 4: life-threatening) during the period of this study. Nanocovax was safe at dose strength of 25 μg, 50 μg and 75 μg.

In phase 2, among 560 participants, there were 554 participants receiving full 2 dose of vaccine or placebo. 6 Withdrew from the study after the 2^{nd} visit, before getting the boosting dose. Local AE were 37.7% after 1st vaccination and 35.6% after 2nd vaccination. Local pain were 32.6% (181/556) after 1st injection and 32.1% (178/554) after 2nd injection. Systemic AEs were 27.4% after 1^{st} injection and 21.8% after 2^{nd} injection. Most common systemic AEs were fatigue (16.9%/13%), headache (13.3%/8.3%) and fever (3.8%/2.4%) after 1^{st}/2^{nd} injections. After 1^{st} injection, local and systemic AE of grade 3 or 4 were not observed in group 2.1, 1 case in group 2.2 (0.6%), 1 case in group 2.3 (0.6%) and 1 case in group 2.4 (1.3%). After 2^{nd} injection, local and systemic AE of grades 3 or 4 were 1 in each vaccine group (0.6%) and placebo (1.3%).

The incidence of unsolicited AEs in were similar in vaccine groups and the placebo. Unsolicited AE incidence of groups 2.1 to 2.4 were 30.4%, 27.5%, 23.3% and 33.8%, respectively. Two cases of grade 4 AE were back pain and dizziness. There were five AE of grade 3: 1 case of sepsis, 1 case of back pain, 1 case of spondylolisthesis, 1 case of sore throat, 1 case of high blood pressure. The most frequently reported adverse events were sore throat 27 (4.8%) and coughing 11 (2%). The most laboratory-related AEs were hyperglycemia 13 cases (2.3%), leukocytosis: 8 cases (1.4%), the most vital events related to hypothermia with 12 cases (2.1%). Similar to phase 1, vaccination pause rules were not implemented in phase 2. Four SAE were determined unrelated to Nanocovax, including 1 case of angina (history of stent graft), 1 case of fever (determined to be sepsis), 1 case of abscess at axillary lymph nodes occurred on the unvaccinated arm and 1 case of personal injury.

The percentage of participants in each vaccine group (groups 2.1, 2.2, 2.3, and 2.4) with adverse events according to the maximum FDA toxicity grade during the 7 days after each vaccination were plotted for solicited local and systemic adverse events. There were no adverse events of grade 4. The most common adverse event was pain at the injection site, mild pain (grade 1) accounted for 23.4% (130/556) after 1st injection and accounted for 26.9% (149/554) after 2nd injection. Moderate pain (grade 2), was uncommon, accounted for 0.4% (2/556) after the 1st injection and 1.1% (6/554) after the 2nd injection. Mild sensitivity at the injection site accounted for 14.7% (82/556) after 1st injection and 14.3% (79/554) after 2nd injection. Moderate sensitivity accounted for 4% (22/556) after injection 1 and 3.8% (21/554) after injection 2. The severe event (grade 3) was “pain at injection site” appearing in 1 participant after 2nd injection, accounting for 0.2% (1/554) and “redness at injection site” occurring in 2 participants (2/554) after 2nd injection. The events mostly occurred within 2 days after vaccination, gradually decreased and disappeared within 7 days after vaccination. Some participants felt fatigue after injection, accounting for 9.4% (52/556) after 1st injection and 8.3% (46/554) after 2nd injection. Fatigue decreased gradually day by day, disappeared at about 7 days after injection. The event “fever” occurred in relatively few participants, mild fever accounted for 0.2% (1/556) after 1st injection and 1.1% (6/554) after 2nd injection. There was 1 participant with high fever (grade 3) accounted for 0.2% (1/556), occurred from day 1 to day 5 after 2nd injection. In the phase 2 study, the proportion of participants with any “unsolicited” AE after injection with vaccine and placebo was 157/560 (28%). The rates of any AE in the study groups were similar between the 25 μg (30.4%), 50 μg (27.5%), 75 μg (23.3%) and placebo (33.8%) groups. Most of “unsolicited” AEs were mild to moderate. When comparing the overall incidence of unsolicited AEs between the vaccine and placebo groups, the rates were similar in the two groups, with a ratio of 130/480 (27.1%) in the vaccinated group compared to the ratio of 27/80 (33.8%) in the placebo group.

Laboratory abnormalities in phase 2 included increased white blood cell in 3 participants, (0.6%), increased neutrophil in 2 participants (0.4%), elevated ALT (grade 2) in 3 participants (0.6%), and elevated AST (grade 2) in 2 participants (0.4%). The other biochemistry and hematology parameters such as red blood cell (RBC), hemoglobin (HGB), creatinine, bilirubin, prothrombin time (PT) fluctuated within normal limits.

### Immunogenicity Outcomes

Anti-S IgG antibody in the serum was quantified by CLIA. Geometric mean concentration (GMC) of anti-S IgG (U/ml) was reported. Before the first injection, the GMC values of the 4 groups were all below the lower limit of detection (0.5 U/ml). Anti-S IgG GMC of 3 vaccine groups increased sharply after 2nd injection, on day 35 and day 42. On day 35, GMC of group 2.1, 2.2 and 2.3 were 6.78 U/ml (95% CI: [5.09-9.03]), 9.38 U/ml [6.99-12.58], and 13.04 U/ml [9.46-17.98] respectively. GMC of group 2.3 was statistically higher than that of group 2.1 but not group 2.2. GMC of the pairs (2.1 vs 2.2) and (2.2 vs. 2.3) were not statistically different. On day 42, GMC of group 2.1, 2.2 and 2.3 increased sharply: 60.48 U/ml [51.12-71.55]; 49.11 U/ml [41.26-58.46] and 57.18 U/ml [48.4-67.5], respectively. The GMC of the placebo group at days 35, and 42 were 0.29 U/ml [0.25-0.33], and 0.29 U/ml [0.25-0.32], respectively.

Geometric mean fold rise (GMFR) of anti-S IgG was defined as the fold increase in GMC of a given timepoint compared to baseline GMC value of the same group on day 0. GMFR of 2.1, 2.2 and 2.3 groups on day 35 were 25.7 [19.3-34.1], 34.7 [ 25.7-46.9], and 49.8 [36.0-68.9], respectively. At day 42, the GMFR of 2.1, 2.2 and 2.3 groups were 229.0 [193.2-271.4], 181.8 [152.0-217.4] and 218.3 [185.0-257.7]. The GMFR of the placebo group (2.4) on days 35 and 42 were 1.05 and 1.04 respectively.

The seroconversion rate was defined as GMFR ≥4. Based on the GMFR of anti-S IgG, the seroconversion rates of 2.1, 2.2 and 2.3 group on day 35 were 84%, 84% and 85%, respectively. On day 42, the seroconversion rate of 2.1, 2.2 and 2.3 groups were 100%, 99% and 100%.

Surrogate virus neutralization test (sVNT) results, expressed as inhibition percentage (%), were considered positive if they were higher than cut-off value of 30%. On day 35, the mean sVNT of groups 2.1 to 2.4 were 58.5% [54.1-63.0], 63.8% [59.1-68.5], 70.2% [65.8-74.5] and 11.1% [9.3-12.7), respectively. The proportions of participants positive for sVNT in groups 2.1 to 2.4 were 80.4%, 82.4%, 86.7% and 1.3%, respectively. On day 42, the mean sVNT of group 2.1 to 2.4 were 87.5% [85.5-89.5], 86.4% [84.08-88.65], 87.1% [85.06-89.16] and 10.8% [8.9-12.67], respectively. Accordingly, the proportions of participants positive for sVNT in groups 2.1 to 2.4 were 100%, 100%, 99.4% and 1.3%, respectively.

Neutralizing antibody titers were evaluated by plaque reduction neutralization test with inhibitory dilution greater than 50% (PRNT₅₀). 112 Serum samples of groups 2.1 to 2.4 were randomly selected for PRNT₅₀ on Wuhan strain and UK variant. The results were expressed as geometric mean titer (GMT). On day 35, GMT of groups 2.1 to 2.3 were 20.9 [12.8-34.1], 22.5 [14.5-34.7] and 33.6 [20.9-54.1], respectively. GMT of group 2.3 was statistically higher than that of group 2.1. No statistical difference was found between the pairs (2.1 vs. 2.2) and (2.2 vs. 2.3). On day 42, GMT of vaccine groups were 89.2 [52.2-152.3], 80 [50.8-125.9] and 95.1 [63.1-143.6] respectively, an almost 3-time increase compared to day 35's. Serum samples of the placebo group at days 35 and 42 did not show neutralizing activity. A small subset of 112 serum samples were randomly selected to test neutralizing activity against UK variant (B.1.1.7, also known as the alpha variant). Neutralizing titer found on UK variant was similar to that of Wuhan strain.

T cell responses of 84 randomly selected participants (28 for each vaccine group and 14 for placebo group) were undetectable. This was likely due to the nature of aluminum adjuvant which has been well established for Th2 response induction.

The results of phase 1 and phase 2 studies demonstrated an excellent safety profile of Nanocovax, regardless of dose strengths. Most adverse events and serious adverse events were grade 1 which disappeared within 48 hours after injection. Compared to similar studies of other approved vaccines, the vaccine may have the least reactogenicity. The vaccine was found to elicit high level of anti-S IgG which closely correlated with neutralizing antibody levels. PRNT results showed that the vaccine, regardless of dose strength, was effective against both original Wuhan strain and UK variant (B.1.1.7). Cellular immune response, evaluated by ICS for IFNg, was not observable. Undetectable IFNg signal, a marker of Th1 response, does not guarantee the absence T cell response. It was likely due to the Th2 promoting nature of aluminum adjuvant. T cell responses may be reevaluated in a small subset of participants in phase 3 with the addition of Th2 cytokines. The Th2 responses, if detected in phase 3, may raise a theoretical concern for vaccine-associated enhanced respiratory disease (ERD). This concern has been partially addressed with the aforementioned SARS-CoV-2 challenge on hamster model.

Although the efficacy of Nanocovax remains to be seen in a phase 3 trial, accumulated evidences have correlated the immunogenicity, particularly the neutralizing antibody level with the protection against Covid-19. Khoury et al. provided a predictive model of efficacy by comparing the neutralizing antibody levels elicited by different vaccines to those of convalescent samples. Khoury D S. et al. Neutralizing antibody levels are highly predictive of immune protection from symptomatic SARS-CoV-2 infection. Nat Med 2021; 27(7):1205-11. Their model suggested that the neutralizing level was highly predictive of immune protection against SARS-CoV-2 infection. Nanocovax is highly safe and immunogenic. Dose strength of 25 μg is selected for phase 3 to evaluate the vaccine efficacy.

While a number of exemplary embodiments, aspects and variations have been provided herein, those of skill in the art will recognize certain modifications, permutations, additions and combinations and certain sub-combinations of the embodiments, aspects and variations. It is intended that the following claims are interpreted to include all such modifications, permutations, additions and combinations and certain sub-combinations of the embodiments, aspects and variations are within their scope. The entire disclosures of all documents cited throughout this application are incorporated herein by reference.

What is claimed is:

1. A modified spike (S) protein comprising of SEQ ID NO: 2.

2. The modified spike protein according to claim 1 encoded by a gene sequence of SEQ ID NO: 1.

3. A gene sequence comprising of SEQ ID NO: 1.

4. The modified spike protein of claim 1, wherein the modified spike protein is cloned with a pCNeoMEM vector and expressed by a host cell line CHO-DG44.

5. An immunogenic composition comprising a modified spike (S) protein comprising of SEQ ID NO: 2; and an adjuvant.

6. The immunogenic composition of claim 5, wherein the modified spike protein is cloned with a pCNeoMEM vector and expressed by a host cell line CHO-DG44.

7. The immunogenic composition of claim 5, wherein the dose comprising the modified spike protein ranges from 5 μg/mL to 100 μg/mL.

8. (canceled)

9. The immunogenic composition of claim 5 further comprising an adjuvant.

10. The immunogenic composition of claim 9 wherein the adjuvant is selected from the group consisting of aluminum phosphate, aluminum hydroxide and saponin QS-21, or a mixture thereof.

11. A method of stimulating an immune response against SARS-CoV-2 or a SARS-CoV-2 variant thereof, in a subject comprising administering a modified spike protein of SEQ ID NO: 2, or a composition comprising a modified spike protein of SEQ ID NO: 2.

12. The method of claim 11, wherein the modified spike protein is encoded by a gene sequence of SEQ ID NO: 1.

13. The method of claim 11, wherein the modified spike protein is cloned with a pCNeoMEM vector and expressed by a host cell line CHO-DG44.

14. The method of claim 11, wherein the SARS-CoV-2 variant is Delta (B.1.617.2).

15. The method of claim 11, wherein the composition comprising a modified spike (S) protein comprising of SEQ ID NO: 2; and an adjuvant.

16. The method of claim 11, wherein the modified spike protein is cloned with a pCNeoMEM vector and expressed by a host cell line CHO-DG44.

17. The method of claim 11, wherein the dose comprising the modified spike protein ranges from 5 μg/mL to 100 μg/mL.

18. The method of claim 11, wherein the dose comprising the modified spike protein is 3 μg/mL or 5 μg/mL.

19. The method of claim 11, wherein the subject is administered a first dose at day 0 and a boost or second dose at day 21; or a boost or second dose at day 30.

20. The method of claim 19, wherein the first dose composition is selected from the group consisting of the modified spike protein of SEQ ID NO: 2, a plasmid DNA encoding the modified spike protein of SEQ ID NO: 2, a viral vector encoding the modified spike protein of SEQ ID NO: 2 and an inactivated modified spike protein of SEQ ID NO: 2.

21. The method of claim 20, wherein the second dose composition is selected from the group consisting of the modified spike protein of SEQ ID NO: 2, a plasmid DNA encoding the modified spike protein of SEQ ID NO: 2, a viral vector encoding the modified spike protein of SEQ ID NO: 2 and an inactivated modified spike protein of SEQ ID NO: 2.
